(19) United States
(12) Patent Application Publication
(10) Pub. No.: US 2023/0363903 A1
(43) Pub. Date: Nov. 16, 2023

(54) MECHANICALLY EXPANDABLE HEART VALVES WITH SEVERAL TYPES OF INTERCONNECTED STRUTS

(71) Applicant: EDWARDS LIFESCIENCES CORPORATION, Irvine, CA (US)

(72) Inventor: Anatoly Dvorsky, Haifa (IL)

(21) Appl. No.: 18/225,069

(22) Filed: Jul. 21, 2023

Related U.S. Application Data
(63) Continuation of application No. PCT/US2022/013509, filed on Jan. 24, 2022.
(60) Provisional application No. 63/141,379, filed on Jan. 25, 2021.

(51) Int. Cl. A61F 2/24

(52) U.S. Cl. CPC A61F 2/2418; A61F 2/90

(57) ABSTRACT

The present invention relates to implantable prosthetic devices, such as mechanically expandable prosthetic heart valves, provided with frames that include more than one type of interconnected struts, wherein each type of struts can extend along a different portion of the frame.

### CROSS REFERENCE TO RELATED APPLICATIONS

This application is a continuation of International Application No. PCT/US2022/013509, filed Jan. 24, 2022, which claims benefit of U.S. Provisional Application No. 63/141,379, filed on Jan. 25, 2021, the contents of each of which are herein incorporated by reference in their entirety.

### FIELD

The present invention relates to implantable prosthetic devices, such as mechanically expandable prosthetic heart valves, provided with frames that include more than one type of interconnected struts, wherein each type of struts can extend along a different portion of the frame.

### BACKGROUND

Native heart valves, such as the aortic, pulmonary and mitral valves, function to assure adequate directional flow from and to the heart, and between the heart's chambers, to supply blood to the whole cardiovascular system. Various valvular diseases can render the valves ineffective and require replacement with artificial valves. Surgical procedures can be performed to repair or replace a heart valve. Conventional surgically implantable prosthetic valve typically include a leaflet assembly mounted within a relatively rigid support frame or ring. Components of the prosthetic valve are usually assembled with one or more biocompatible fabrics, and a fabric-covered sewing ring is provided around the valve for suturing to the tissue of the native leaflet.

One of the problems encountered with existing surgically implantable heart valves is that it can be difficult to select a valve that is properly sized to fir into the native annulus of a patient. Currently, surgically implantable valves may be provided in several distinct sizes, each of which for use with a specific range of diameters of the native annulus. However, it can be difficult to select a prosthetic valve that is properly sized to accurately fit into the specific annulus of a patient. A prosthetic valve having the closest diameter to that of the annulus, yet smaller than the actual annular diameter if an exact match is not possible, is conventionally selected, which may often result in patient prosthetic mismatch (PPM). This may in turn result in higher transvalvular pressure gradients, and is associated with decreased regression of left ventricular hypertrophy, reduced coronary flow reserve, increased incidence of congestive heart failure, diminished functional capacity, and increased risk of early and late mortality. It would be desirable, therefore, to provide a prosthetic valve that is resizable pre-implantation, such that the diameter of the surgically implantable valve can be adjusted to properly match the patient's annular size.

While stitching a suturing ring of a surgically implantable valve to the native annular tissue may provide adequate desired retention of the valve in position over the course of several years, surgeries are prone to an abundance of clinical complications, hence alternative less invasive techniques of delivering a prosthetic heart valve over a catheter and implanting it over the native malfunctioning valve, have been developed over the years.

One exemplary technique includes utilization of a delivery assembly for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter. Due to the inability to suture such valves to the native tissue, as opposed to surgically implantable valves, there is a need for improvements to devices and methods for the ability of the prosthetic heart valve to remain at the treatment location after deployment without becoming dislodged.

### SUMMARY

The present disclosure is directed toward prosthetic heart valves that include an expandable frame and a leaflet assembly mounted therein, wherein the frame includes at least two types of struts pivotably interconnected, such that struts of each type extend along a different portion of the frame between its inflow end and outflow end.

According to an aspect of the invention, there is provided a prosthetic valve comprising a frame movable between a range of diameters. The frame extends between an inflow end and an outflow end and comprises a plurality of struts, wherein each strut comprises a plurality of connection portions and segments extending between the connection portions. The segments of each strut comprise a proximal-most segment and at least one subsequent segment. The plurality of struts are pivotably interconnected at their connection portions, thereby defining a plurality of junctions.

The plurality of junctions comprises a plurality of outflow apices along the outflow end, a plurality of inflow apices along the inflow end, and a plurality of non-apical junctions between the outflow end and the inflow end. The plurality of struts comprises a plurality of first-type struts and a plurality of second-type struts. Each strut is curved helically with respect to a longitudinal axis of the frame. The first-type strut is longer than the second-type strut. The first-type struts extend distally from the outflow end, and the second-type struts extend distally from non-apical junctions which are distal to the outflow end.

According to some examples, each first-type strut comprises three segments, wherein the proximal-most segment of the first-type strut is longer than any of its subsequent segments.

According to some examples, the segments subsequent to the proximal-most segment of the first-type strut are having progressively shorter lengths.

According to some examples, each second-type strut comprises three segments, wherein the proximal-most segment of the second-type strut is longer than any of its subsequent segments, and wherein the proximal-most segment of the first-type strut is longer than any of the segments of the second-type strut.

According to some examples, the segments subsequent to the proximal-most segment of the second-type strut are having progressively shorter lengths.

According to some examples, each second-type strut comprises two segments, wherein the proximal-most segment is longer than the subsequent segment.

According to some examples, the struts are concave with respect to the outflow end.

According to some examples, the frame has a draft angle that increases when the valve is expanded to a larger diameter of the outflow end.

According to some examples, the first-type struts extend from the outflow apices to non-apical junctions.

According to some examples, the first-type struts extend from the outflow apices to distal-most non-apical junctions.

According to some examples, pairs of second-type struts intersect at the inflow apices and extend therefrom to non-apical junctions.

According to some examples, the second-type struts extend from the inflow apices to proximal-most non-apical junctions.

According to some examples, the prosthetic valve further comprises a leaflet assembly mounted within the frame. The leaflet assembly comprises a plurality of leaflets configured to regulate flow through the prosthetic valve, wherein each leaflet comprises a pair of oppositely-directed tabs, and wherein the tabs of adjacent leaflets are paired to form commissures.

According to some examples, the prosthetic valve further comprises a plurality of support members, wherein each support member is attached to four struts defining a commissure cell along a first row of cells of the frame, and wherein each commissure is attached to a respective support member.

According to some examples, each support member covers the entire area of the corresponding commissure cell.

According to some examples, each segment of the first-type strut is curved with respect to a first lateral axis.

According to some examples, the proximal-most segment of each first-type strut comprises a proximal portion extending from the outflow end, and wherein each commissure is attached to a pair of proximal portions.

According to some examples, each pair of adjacent proximal portions defines a commissure angle that is not greater than 30 degrees at the maximal diameter of the prosthetic valve.

According to some examples, the commissure angle is not greater than 15 degrees at the maximal diameter of the prosthetic valve.

According to some examples, the commissure angle is not greater than 10 degrees at the maximal diameter of the prosthetic valve.

According to some examples, the commissure angle is not greater than 5 degrees at the maximal diameter of the prosthetic valve.

According to some examples, the length of the proximal portion is at least as great as a quarter of the length of the proximal-most segment of the first-type strut.

According to some examples, the length of the proximal portion is at least as great as a third of the length of the proximal-most segment of the first-type strut.

According to some examples, the length of the proximal portion is at least as great as half of the length of the proximal-most segment of the first-type strut.

According to some examples, pairs of the first-type struts intersect at the outflow apices.

According to some examples, each pair of adjacent proximal portions are distanced from each other at the outflow end.

According to some examples, the distance between each pair of adjacent proximal portions is not greater than four times the thickness of the tab of the leaflet.

According to some examples, the distance between each pair of adjacent proximal portions is not greater than twice the thickness of the tab of the leaflet.

According to some examples, the plurality of non-apical junctions includes a plurality of proximal-most non-apical junctions, wherein exactly three segments extend from each proximal-most non-apical junction.

According to some examples, the plurality of non-apical junctions includes a plurality of distal-most non-apical junctions, wherein exactly three segments extend from each distal-most non-apical junction.

According to some examples, a sewing ring that circumscribes the frame.

According to some examples, the sewing ring comprises a ring insert and a cloth cover around the ring insert.

According to some examples, the prosthetic valve further comprises a skirt mounted on the outer surface of the frame, wherein the sewing ring is sutured to the skirt.

According to some examples, the prosthetic valve further comprises a plurality of expansion and locking assemblies, wherein each expansion and locking assembly is secured to the frame at a first location and a second location that is axially spaced from the first location, and configured to facilitate expansion or contraction of the prosthetic valve by approximating the first and second locations to each other, or distancing them away from each other, respectively.

According to some examples, the plurality of expansion and locking assemblies comprises three expansion and locking assemblies.

According to some examples, each expansion and locking assembly comprises an outer member and an inner member. The outer member is secured to the frame at the first location, and comprises a spring-biased arm with a pawl. The inner member is secured to the frame at the second location spaced apart from the first location, and extends at least partially into the outer member. The inner member comprises a plurality of ratcheting teeth. The spring-biased arm is biased toward the inner member. Engagement of the pawl with the ratcheting teeth allows movement in a first direction to allow axial foreshortening and radial expansion of the frame, and prevents movement in a second direction to prevent radial compression of the frame

According to some examples, each expansion and locking assembly comprises a first anchor, a second anchor, and a rod. The first anchor is secured to the frame at the first location, and defines a first anchor channel. The second anchor is secured to the frame at the second location, and defines a second anchor threaded channel. The rod extends through the first anchor channel and the second anchor threaded channel, and is threaded through the second anchor threaded channel such that rotation of the rod causes corresponding axial movement of the second anchor toward or away from the first anchor, depending on the direction of rotation.

According to another aspect of the invention, there is provided a prosthetic valve comprising a frame movable between a range of diameters. The frame extends between an inflow end and an outflow end and comprises a first row of cells comprising a plurality of commissure cells, and a second row of cells, wherein the number of cells in the second row of cells is higher than the number of cells in the first row of cells.

According to some examples, wherein the first row of cells comprises three cells, and wherein the second row of cells comprises six cells.

According to some examples, the height of the first row of cells is greater than the height of the second row of cells.

According to some examples, the prosthetic valve further comprises a third row of cells, wherein the number of cells in the second row of cells is higher than the number of cells in the third row of cells.

According to some examples, the third row of cells comprises three cells.

According to some examples, the height of the second row of cells is greater than the height of the third row of cells.

According to some examples, the frame has a draft angle that increases when the valve is expanded to a larger diameter of the outflow end.

According to some examples, the prosthetic valve further comprises a leaflet assembly mounted within the frame. The leaflet assembly comprises a plurality of leaflets configured to regulate flow through the prosthetic valve, wherein each leaflet comprises a pair of oppositely-directed tabs, and wherein the tabs of adjacent leaflets are paired to form commissures.

According to some examples, the prosthetic valve further comprises a plurality of support members, wherein each support member is attached to a cell of the first row of cells, and wherein each commissure is attached to a respective support member.

According to some examples, each support member covers the entire area of the corresponding cell it is attached to.

According to some examples, each cell of the first row of cells comprises a pair of proximal portions extending from the outflow end, and wherein each commissure is attached to the corresponding pair of proximal portions.

According to some examples, each pair of adjacent proximal portions defines a commissure angle that is not greater than 30 degrees at the maximal diameter of the prosthetic valve.

According to some examples, the commissure angle is not greater than 15 degrees at the maximal diameter of the prosthetic valve.

According to some examples, the commissure angle is not greater than 10 degrees at the maximal diameter of the prosthetic valve.

According to some examples, the commissure angle is not greater than 5 degrees at the maximal diameter of the prosthetic valve.

According to some examples, the prosthetic valve further comprises a sewing ring that circumscribes the frame.

According to some examples, the sewing ring comprises a ring insert and a cloth cover around the ring insert.

According to some examples, the prosthetic valve further comprises f a skirt mounted on the outer surface of the frame, wherein the sewing ring is sutured to the skirt.

According to some examples, the prosthetic valve further comprises a plurality of expansion and locking assemblies, wherein each expansion and locking assembly is secured to the frame at a first location and a second location that is axially spaced from the first location, and configured to facilitate expansion or contraction of the prosthetic valve by approximating the first and second locations to each other, or distancing them away from each other, respectively.

According to some examples, the plurality of expansion and locking assemblies comprises three expansion and locking assemblies.

According to some examples, each expansion and locking assembly comprises an outer member and an inner member. The outer member is secured to the frame at the first location, and comprises a spring-biased arm with a pawl. The inner member is secured to the frame at the second location spaced apart from the first location, and extends at least partially into the outer member. The inner member comprises a plurality of ratcheting teeth. The spring-biased arm is biased toward the inner member. Engagement of the pawl with the ratcheting teeth allows movement in a first direction to allow axial foreshortening and radial expansion of the frame, and prevents movement in a second direction to prevent radial compression of the frame.

According to some examples, each expansion and locking assembly comprises a first anchor, a second anchor, and a rod. The first anchor is secured to the frame at the first location, and defines a first anchor channel. The second anchor is secured to the frame at the second location, and defines a second anchor threaded channel. The rod extends through the first anchor channel and the second anchor threaded channel, and is threaded through the second anchor threaded channel such that rotation of the rod causes corresponding axial movement of the second anchor toward or away from the first anchor, depending on the direction of rotation.

According to yet another aspect of the invention, there is provided a prosthetic valve comprising a frame movable between a compressed configuration and an expanded configuration. The frame extends between an inflow end and an outflow end and comprises a plurality of struts, wherein each strut comprises a plurality of connection portions and segments extending between the connection portions. The segments of each strut comprise a proximal-most segment and at least one subsequent segment. The plurality of struts are pivotably interconnected at their connection portions, thereby defining a plurality of junctions.

The plurality of junctions comprises a plurality of outflow apices along the outflow end, a plurality of inflow apices along the inflow end, and a plurality of non-apical junctions between the outflow end and the inflow end. The plurality of struts comprises a plurality of first-type struts, a plurality of second-type struts, and a plurality of third-type struts. The first-type struts extend from the inflow end to the outflow end. The second-type struts extend from the inflow end to non-apical junctions distal to the outflow end.

Each third-type strut comprises a proximal segment and a distal segment, wherein pairs of distal segments of the third-type struts are interconnected at a clamp apices, each pair of interconnected distal segments defining a clamping portion. The clamping portions are movable between an open position corresponding to the compressed configuration of the frame and a closed position corresponding to the expanded configuration of the frame. Motion of the frame between the compressed configuration and the expanded configuration causes corresponding motion of the clamping portions between the open position and the closed position.

According to some examples, each proximal segment of a third-type strut is connected to two first-type struts at an outflow apex and a non-apical junction.

According to some examples, the clamp apices are free ends that are not connected to any of the first-type struts or the second-type struts, such that when the clamping portions are in the open position, the clamp apices are spaced radially outwardly from the remainder of the frame.

According to some examples, the clamp apices are proximal to the inflow end in the compressed configuration.

According to some examples, the number of second-type struts is identical to the number of third-type struts, wherein each second-type strut is diagonally aligned with a corresponding proximal segment of a third-type strut.

According to some examples, the segments of the first-type strut are linear.

According to some examples, all of the segments of the first-type strut have identical lengths.

According to some examples, the segments of the second-type strut are linear.

According to some examples, all of the segments of the first-type strut have identical lengths.

According to some examples, the number of segments of the first type strut is larger than the number of segments of the second type strut.

According to some examples, the first-type strut comprises six segments, and wherein the second-type strut comprises three segments.

According to some examples, all of the segments of the first-type strut and all of the segments of the second-type struts have identical lengths.

According to some examples, the length of the proximal segment of the third-type strut is equal to the length of a proximal-most segment of the first-type strut.

According to some examples, the proximal segment of the third-type strut is linear.

According to some examples, none of the second-type struts contacts any of the third-type struts in any configuration of the frame.

According to some examples, the inflow apices and the outflow apices are offset radially outward to the non-apical junctions in the expanded configuration, such that the portion of the frame that includes the first-type struts and the second-type struts has an hourglass-shaped profile.

According to some examples, the inflow apices and the outflow apices are offset radially inward to the non-apical junctions in the compressed configuration, such that the portion of the frame that includes the first-type struts and the second-type struts has a barrel-shaped profile.

According to some examples, the distal segment of the third-type strut has a projected length that is greater than the length of the proximal segment of the third-type strut.

According to some examples, the projected length of the distal segment is shorter than twice the length of the proximal segment.

According to some examples, the third-type struts are dimensioned such that the clamping portions can reside at the level of the surface defined by the first-type struts and the second-type struts.

According to some examples, the projected length of the distal segment is greater than twice the length of the proximal segment.

According to some examples, the projected length of the distal segment is greater than at least three times the length of the proximal segment.

According to some examples, the distal segment of the third-type strut is curved.

According to some examples, the distal segment of the third-type strut is linear.

According to some examples, the proximal segment and the distal segment form an angle there-between that is not greater than 170 degrees.

According to some examples, the prosthetic valve further comprises a leaflet assembly mounted within the frame. The leaflet assembly comprises a plurality of leaflets configured to regulate flow through the prosthetic valve, wherein each leaflet comprises a pair of oppositely-directed tabs, and wherein the tabs of adjacent leaflets are paired to form commissures

According to some examples, the prosthetic valve further comprises a plurality of expansion and locking assemblies, wherein each expansion and locking assembly is secured to the frame at a first location and a second location that is axially spaced from the first location, and configured to facilitate expansion or contraction of the prosthetic valve by approximating the first and second locations to each other, or distancing them away from each other, respectively.

According to some examples, the plurality of expansion and locking assemblies comprises three expansion and locking assemblies.

According to some examples, each expansion and locking assembly comprises an outer member and an inner member. The outer member is secured to the frame at the first location, and comprises a spring-biased arm with a pawl. The inner member is secured to the frame at the second location spaced apart from the first location, and extends at least partially into the outer member. The inner member comprises a plurality of ratcheting teeth. The spring-biased arm is biased toward the inner member. Engagement of the pawl with the ratcheting teeth allows movement in a first direction to allow axial foreshortening and radial expansion of the frame, and prevents movement in a second direction to prevent radial compression of the frame.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 is a perspective view of a conventional surgically implantable prosthetic valve with portions cutaway to reveal internal structural components thereof, according to some examples.

FIG. 2 is a view in perspective of a mechanically expandable surgically implantable prosthetic valve, according to some examples.

FIGS. 3A-3B show a frame of the prosthetic valve of FIG. 2 in two expansion configurations, according to some examples.

FIGS. 4A-4B show flattened projections of two types of struts, according to some examples.

FIG. 5 shows a frame of a mechanically expandable surgically implantable prosthetic valve, equipped with a plurality of stages of ratcheting-type expansion and locking assemblies, according to some examples.

FIG. 6 shows a frame of a mechanically expandable surgically implantable prosthetic valve, equipped with a plurality of stages of threaded-type expansion and locking assemblies, according to some examples.

FIG. 7 shows a frame of another type of a mechanically expandable surgically implantable prosthetic valve, according to some examples.

FIG. 8 shows a flattened projection of another type of strut, according to some examples.

FIGS. 9A-9B show views in perspective of another type of a mechanically expandable surgically implantable prosthetic valve, illustrated with and without soft components thereof, according to some examples.

FIG. 10 shows a flattened projection of another type of strut, according to some examples.

FIGS. 11A-11B show views in perspective of a mechanically expandable prosthetic valve equipped with connection portions, illustrated with and without soft components thereof, according to some examples.

FIGS. 12A-12C show flattened projections of three types of struts, according to some examples.

FIG. 13A is a side elevation view of one layer of struts of a frame, wherein the struts are arranges in the shape of a prosthetic valve at a natural diameter of the frame.

FIG. 13B is a side elevation view illustrating a position of the struts when the frame of FIG. 13A is radially compressed to a diameter that is less than its natural diameter.

FIG. 13C is a side elevation view illustrating a position of the struts when the frame of FIG. 13A is radially expanded to a diameter that is larger than its natural diameter.

FIG. 14 is a side elevation view illustrating the frame of FIG. 11B in a compressed configuration in a native aortic valve, and including connection portions in an open position.

FIG. 15 is a side elevation view illustrating the frame of FIG. 11B in an expanded configuration in a native aortic valve, such that the connection portions are in a closed position, clamped over the native leaflets.

FIG. 16 is a side elevation view illustrating the frame of FIG. 11B in a compressed configuration with the connection portions in the open position, according to some examples.

FIG. 17 is a side elevation view illustrating the frame of FIG. 11B in an expanded configuration with the connection portions in the closed position, according to some examples.

FIG. 18 shows a flattened projection of another type of strut, according to some examples.

FIG. 19 shows a frame of another type of a mechanically expandable prosthetic valve equipped with connection portions, according to some examples.

FIG. 20 shows a flattened projection of another type of strut, according to some examples.

FIG. 21 shows a frame of another type of a mechanically expandable prosthetic valve equipped with connection portions, according to some examples.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the terms “have” or “includes” means “comprises.” As used herein, “and/or” means “and” or “or,” as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,” “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

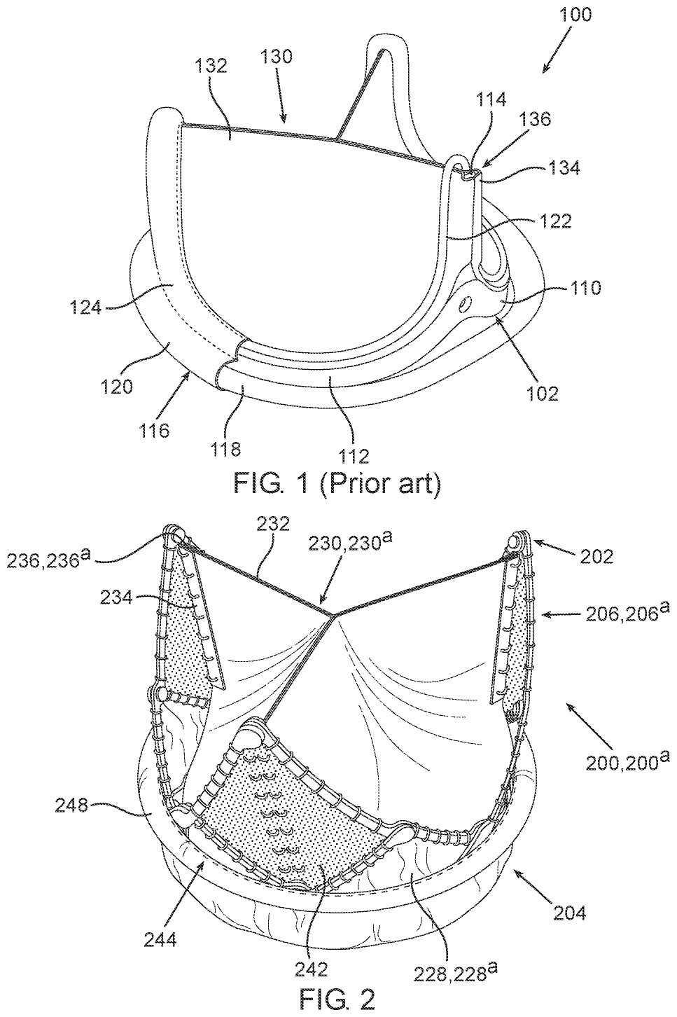

FIG. 1 shows an example of a surgically implantable prosthetic valve 100. In the particular example depicted, the valve 100 comprises a support frame 102 and a leaflet assembly 130 attached thereto. The leaflet assembly 130 comprises a plurality of leaflets 132 (e.g., three leaflets), positioned at least partially within the frame 102, and configured to regulate flow of blood through the prosthetic valve 100. While three leaflets 132 arranged to collapse in a tricuspid arrangement similar to the native aortic valve, are shown in the example illustrated in FIG. 1, it will be clear that a prosthetic valve 100 can include any other number of leaflets 132, such as two leaflets configured to collapse in a bicuspid arrangement similar to the native mitral valve, or more than three leaflets, depending upon the particular application. The leaflets 132 are made of a flexible material, derived from biological materials (e.g., bovine pericardium or pericardium from other sources), bio-compatible synthetic materials, or other suitable materials as known in the art and described, for example, in U.S. Pat. Nos. 6,730,118, 6,767,362 and 6,908,481, which are incorporated by reference herein.

The term “plurality”, as used herein, means more than one.

The terms coupled, engaged, connected and attached, as used herein, are interchangeable.

The frame 102 comprises a band 110 which is generally rigid and/or expansion-resistant in order to maintain the particular shape and diameter of the valve 100, and a plurality of commissure posts 114 (e.g., three posts) extending proximally from the band 110 to support the free edges of the leaflets 132. The band 110 can comprise cusp portions 112 extending between the vertically oriented commissure posts 114. The frame 102 can be metallic, plastic, or a combination of the two.

The term “proximal”, as used herein, generally refers to a position, direction, or portion of any device or a component of a device, which is closer to the user (e.g., clinician) and further away from the implantation site.

The term “distal”, as used herein, generally refers to a position, direction, or portion of any device or a component of a device, which is further away from the user (e.g., clinician) and closer to the implantation site.

The term “outflow”, as used herein, refers to a region of the prosthetic valve through which the blood flows through and out of the valve.

The term “inflow”, as used herein, refers to a region of the prosthetic valve through which the blood flows into the valve.

In some configurations, as illustrated, the surgically implantable valve 100 further comprises an undulating wireform 122, configured to provide further support to the leaflets 132. The wireform 122 can include a plurality (e.g., three) large radius wireform cusps supporting the cusp regions of the leaflet assembly 130, while the ends of each pair of adjacent wireform cusps converge somewhat asymptotically to form upstanding wireform commissure portions that terminate in tips, each extending in the opposite direction as the arcuate wireform cusps and having a relatively smaller radius. The cusp portions 112 and the commissure posts 114 can be sized and shaped so as to correspond to the curvature of the wireform 122.

Wireform 122 typically is formed from one or more pieces of wire but also can be formed from other similarly-shaped elongate members. The wireform can also be cut or otherwise formed from tubing or a sheet of material. The wireform can have any of various cross sectional shapes, such as a square, rectangular, circular, or combinations thereof. In some examples, wireform 122 is made of a relatively rigid metal, such as stainless steel or Elgiloy (a Co—Cr—Ni alloy). In some examples, the wireform 122 further comprises a wireform cloth encapsulating it along its length.

Each of the leaflets 132 can be attached along a cusp edge thereof to a corresponding cusp portion 112 of the band 110 and up along adjacent commissure posts 114. Each leaflet 132 can include a pair of oppositely-directed tabs 134, wherein each respective tab 134 can be aligned with a tab 134 of an adjacent leaflet 132 as shown. Each pair of aligned tabs 134 can be inserted between adjacent upstanding wireform portions. The tabs 134 can then be wrapped around a respective commissure posts 114 of the frame 102. The tabs 134 can be sutured or otherwise coupled to each other and/or to the commissure post 114, thereby forming commissure assemblies 136 that project in an outflow direction along the longitudinal axis 10 of the valve. The wireform 122 and the commissure posts 114 provide flexibility to the commissure assemblies 136 which helps reduce stress on the bioprosthetic material of the leaflets 132.

A soft sealing or sewing ring 116 circumscribes the frame 102, for example around the band 110, and is typically used to secure the valve to a native annulus such as with sutures. The sewing ring 116 comprises a sewing ring insert 118 and a cloth cover 120. The sewing ring insert 118 can be made of a suture permeable material for suturing the valve to a native annulus, as known in the art. For example, the sewing ring insert 118 can be made of a silicone-based material, although other suture-permeable materials can be used. The cloth cover 120 can be formed of any biocompatible fabric, such as, for example, polyethylene terephthalate or polyester fabric.

A skirt 124 can completely cover the frame 102, including the band 110 and the commissure posts 114. The wireform is secured to the inner side of the frame 102, wherein the skirt 124 can, in some examples, cover the wireform 122 as well. The sewing ring 116 can be secured to the frame 102 by being stitched to the skirt 124, or via sutures extending through the sewing ring 116 and apertures of the band 110. The skirt 124 can be formed of any biocompatible fabric, such as, for example, polyethylene terephthalate or polyester fabric.

The terms “including” and/or “having”, as used herein, are defined as comprising (i.e., open language).

Conventional surgically implantable prosthetic valves, such as valve 100 described hereinabove and illustrated in FIG. 1, may be provided in several distinct sizes, each of which for use with a specific range of diameters of the target implantation annulus. However, it can be difficult to select a prosthetic valve that is properly sized to fit into the specific annulus (e.g., aortic annulus) of a patient. Currently, a prosthetic valve having the closest diameter to that of the annulus, yet smaller than the actual annular diameter if an exact match is not possible, is selected, which may often result in patient prosthetic mismatch (PPM). This may in turn result in higher transvalvular pressure gradients, and is associated with decreased regression of left ventricular hypertrophy, reduced coronary flow reserve, increased incidence of congestive heart failure, diminished functional capacity, and increased risk of early and late mortality. It would be desirable, therefore, to provide a prosthetic valve that is resizable pre-implantation, such that the diameter of the surgically implantable valve can be adjusted to properly match the patient's annular size.

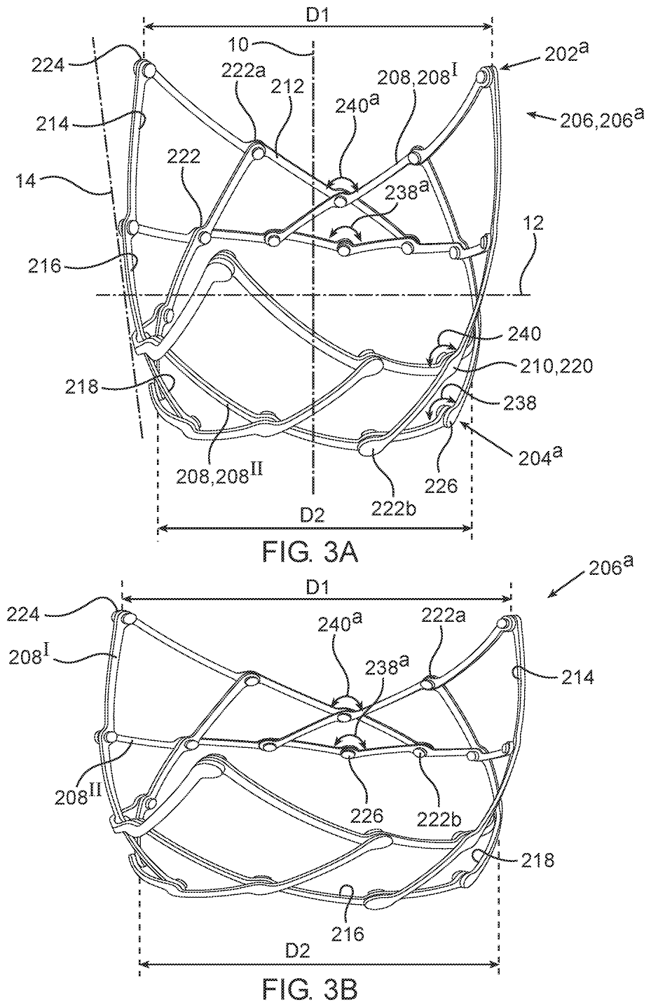

FIGS. 2-3B show perspective views a mechanically-expandable surgically implantable prosthetic valve 200 that can be size-adjusted to a desired diameter pre-implantation, shown with (FIG. 2) and without (FIGS. 3A-B) soft components (such as a skirt, a sewing ring and a leaflet assembly). A specific example of a mechanically expandable surgically implantable prosthetic valve 200^{a} is illustrated in FIGS. 2-3A in a first diameter, and in FIG. 3B further expanded to a larger diameter. Mechanically expandable valves are a category of prosthetic valves that rely on a mechanical actuation mechanism for expansion. The mechanical actuation mechanism usually includes a plurality of expansion and locking assemblies, which may be actuated by an actuation apparatus (not shown) to expand the prosthetic valve to a desired diameter.

The prosthetic valve 200 can comprise an inflow end 204 and an outflow end 202. In some instances, the outflow end 202 is the distal end of the prosthetic valve 200, and the inflow end 204 is the proximal end of the prosthetic valve 200. Alternatively, the outflow end can be the proximal end of the prosthetic valve, and the inflow end can be the distal end of the prosthetic valve. The prosthetic valve 200 can define a longitudinal axis 10 extending from the inflow end 204 to the outflow end 202 and a lateral axis 12 extending perpendicular to the longitudinal axis 10.

The prosthetic valve 200 comprises an annular frame 206 movable between a range of diameters, and a leaflet assembly 230 mounted within the frame 206. The frame 206 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. The frame can include a plurality of interconnected struts 208 arranged in a lattice-type pattern. The struts are shown as positioned diagonally, or offset at an angle relative to, and radially offset from, the longitudinal axis 10 of the valve. In other implementations, the struts 208 can be offset by a different amount than depicted in FIGS. 2-3B.

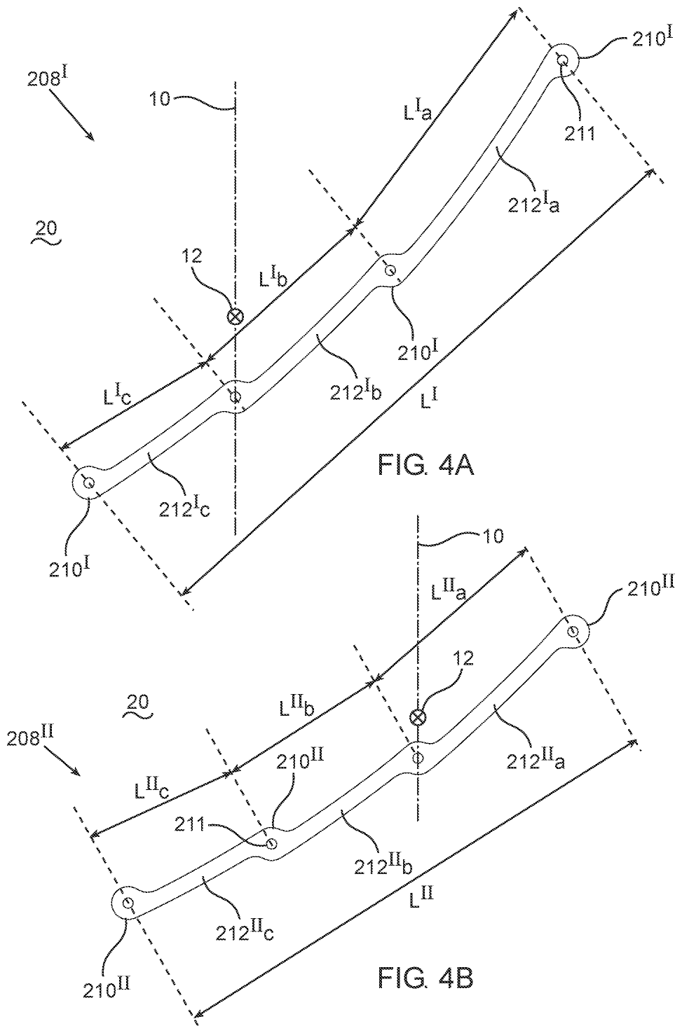

In the illustrated example, the struts 208 are pivotably coupled to one another at one or more pivot joints along the length of each strut. For example, in the illustrated configuration, each of the struts 208 can be formed with apertures 211 (see e.g., FIG. 4A) at opposing ends of the strut and apertures 211 spaced along the length of the strut. Respective hinges can be formed at the locations where struts 208 overlap each other via fasteners, such as rivets or pins that extend through the apertures. The hinges can allow the struts 208 to pivot relative to one another as the frame 206 is radially expanded or compressed, such as during assembly or pre-implantation size-adjustment of the prosthetic valve 200.

The pivot joints of interconnected struts 208 form junctions 220 of the valve 200, that can include outflow apices 224 at the outflow end 202 of the valve 200, inflow apices 226 at the inflow end 204 of the valve 200, and other non-apical junctions 222, such as the proximal-most non-apical junctions 222a and the distal-most non-apical junctions 222b shown in FIGS. 3A-B.

In some examples, the frame 206 (or frame 506 described further below) can be constructed by forming individual components (e.g., the struts and fasteners of the frame) and then mechanically assembling and connecting the individual components together. In other examples, the struts 208 are not coupled to each other with respective hinges but are otherwise pivotable or bendable relative to each other to permit radial expansion and contraction of the frame 206. For example, the frame 206 can be formed (e.g., via laser cutting, electroforming or physical vapor deposition) from a single piece of material (e.g., a metal tube). Further details regarding the construction of the frame and the prosthetic valve are described in U.S. Publication Nos. 2018/0153689; 2018/0344456; 2019/0060057 all of which are incorporated herein by reference.

The leaflet assembly 230 comprises a plurality of leaflets 232 (e.g., three leaflets), positioned at least partially within the frame 206, and configured to regulate flow of blood through the prosthetic valve 200 from the inflow end 204 to the outflow end 202. While three leaflets 232 arranged to collapse in a tricuspid arrangement similar to the native aortic valve, are shown in the exemplary example illustrated in FIG. 2, it will be clear that a prosthetic valve 200 can include any other number of leaflets 232, such as two leaflets configured to collapse in a bicuspid arrangement similar to the native mitral valve, or more than three leaflets, depending upon the particular application. Similar to leaflets 232 described hereinabove with respect to surgically implantable valve 100, the leaflets 232 are made of a flexible material, derived from biological materials (e.g., bovine pericardium or pericardium from other sources), bio-compatible synthetic materials, or other suitable materials as known in the art and described, for example, in U.S. Pat. Nos. 6,730,118, 6,767,362 and 6,908,481, which are incorporated by reference herein.

According to some examples, the prosthetic valve 200 further comprises a skirt 228 that can be mounted on the outer surface of the frame 206. The skirt 228 can be connected to the frame 206 using suitable techniques or mechanisms. For example, the skirt 228 can be sutured to the frame 206 via sutures that can extend around the struts 208. The skirt 228 can be made of various suitable biocompatible materials, such as, but not limited to, various synthetic materials (e.g., PET) or natural tissue (e.g. bovine pericardium).

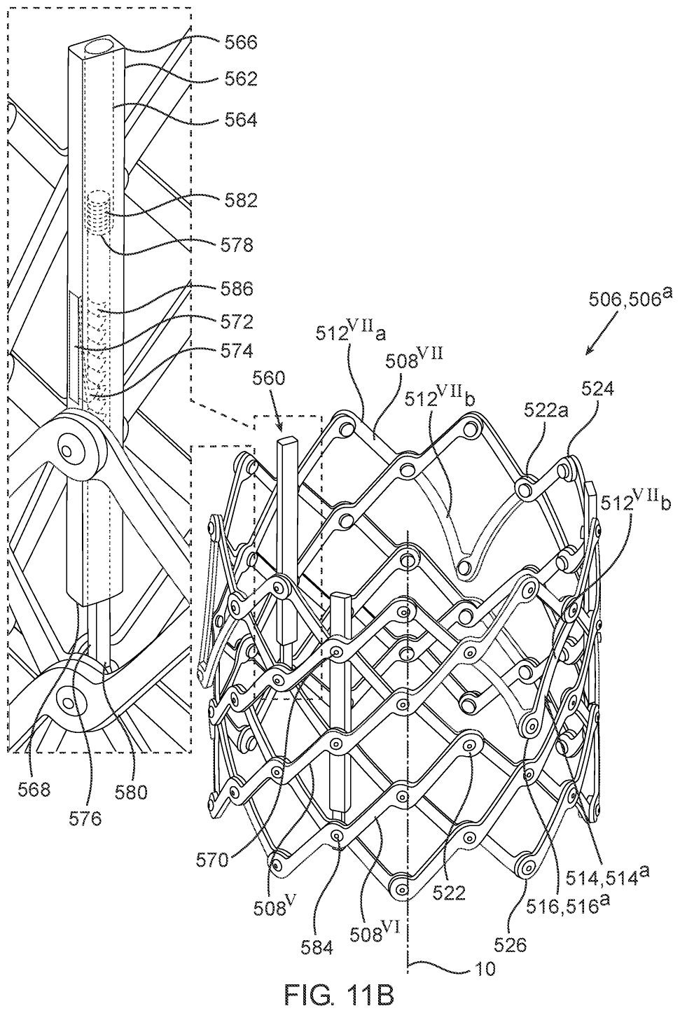

The inflow or cusp edges of the leaflets 232 can be secured to the frame 206 using one or more connecting skirts (not shown). In alternative examples, the cusp edges of the leaflets 232 can be directly coupled, for example via a series of suture stitches, to the struts 208 of a frame 206, or to other types of connecting fabrics such as an inner skirt mounted over the inner surface of the frame, which is not shown in FIG. 3A but may be similar to inner skirt 528 described below in conjunction with FIG. 11A. For example, the leaflet assembly 230 can be sutured to an inner skirt along a scallop line (not shown) that tracks the lower edge of the leaflet assembly 230. Further details regarding prosthetic valves, including the manner in which leaflets may be mounted to their frames, are described in U.S. Pat. Nos. 7,393,360, 7,510,575, 7,993,394 and 8,252,202, US Pat. Publication No. 2018/0028310, and U.S. Patent Application No. 62/614,299, all of which are incorporated herein by reference.

Each leaflet 232 comprises opposing tabs 234. Each tab 234 can be secured to an adjacent tab 234 of an adjacent leaflet 232 to form a commissure 236 that is secured to the frame 206. The tabs 234 can be folded in various manners, for example to form radially extending layers and circumferentially extending layers facing the frame. Radially extending layers can extend radially inward, from a location on the frame 206 toward longitudinal axis 10.

During valve cycling, the leaflets 232 can articulate at the inner most edges of the tab layers, which helps space the leaflets away from the frame 206 during normal operation of the prosthetic valve 200. This is particular advantageous in cases where the prosthetic valve 200 is not fully expanded to its nominal size prior to and/or when surgically implanted in a patient. As such, the prosthetic valve 200 can be sized to be used in a wider range of patient annulus sizes.

Each strut 208 can include a plurality of connection portions 210 at which it may be interconnected with another strut to define the junctions 220, the connection portions 210 defining segments 212 extending there-between. The connection portions 210 can be formed as enlarged (relative to the segments 212) end portions or enlarged intermediate portions. In some examples, struts 208 include a plurality of apertures 211, each aperture 211 comprised within a connection portion, as shown in FIGS. 4A-B. In other examples, a strut can include other coupling means, such as integrally formed pins (not shown) extending from corresponding connection portions 210, that may be configured to be received within apertures 211 of struts 208 interconnected therewith.

In some examples, as further shown in FIGS. 4A-B, the connection portions 210 can be spaced unequally along the length of each strut 208, defining a plurality of segments 212 having unequal lengths. Frame 206 of a mechanically-expandable surgically implantable valve 200 comprises at least two types of interconnected struts 208, namely first-type struts and second-type struts, each type having a different length, and each type extending along a different portion of the frame 206 between the inflow end 204 and the outflow end 202. The first-type struts intersect with each other to form outflow apices 224 along the outflow end 202, and extend distally therefrom such that they may either terminate at inflow apices 226 along the inflow end 204, or at non-apical junctions 222 proximal to the inflow end. The second-type struts do not reach the outflow end 202, but rather extend distally from non-apical junctions 222 which are distal to the outflow end 202, and may either terminate at inflow apices 226 along the inflow end 204, or at non-apical junctions 222 proximal to the inflow end.

In some examples, each of the first-type struts and the second-type struts are provided with a different number of connection portions 210, thereby defining a different number of segments 212. In other examples, each of the first-type struts and the second-type struts are provided with same number of connection portions 210 defining the same number of segments 212, yet the length of at least one of the segments of the first-type is different from the length of any of the segments of the second-type strut.

FIGS. 4A and 4B show flattened projections of two types of struts 208 in a plane 20 parallel to the longitudinal axis 10 of the valve 200. As shown, the lateral axis 12 is perpendicular to the longitudinal axis 10 and the plane 20. As further shown for both types of struts 208, each segment 212 can be slightly laterally offset from an adjacent segment 212 in a direction perpendicular to the overall length of the strut 208. In alternative examples, the segments 212 can be arranged without any offset relative to each other.

FIG. 4A shows a flattened projection of an example of a strut 208^{I} comprising three segments 212^{I}a, 212^{I}b, and 212^{I}c, with segment 212^{I}a, which can be the proximal-most segment of the strut 208^{I}, being the longest, and each subsequent segment 212^{I}b, and 212^{I}c having a progressively shorter length. For example, strut 208^{I} can have a total length L^{I}, and the segments 212^{I}a, 212^{I}b, and 212^{I}c can have corresponding lengths L^{I}a, L^{I}b, and L^{I}c. In the example illustrated in FIG. 4A, L^{I}a>L^{I}b>L^{I}c.

FIG. 4B shows a flattened projection of another example of a strut 208^{II} comprising three segments 212^{II}a, 212^{II}b, and 212^{II}c, with segment 212^{II}a, which can be the proximal-most segment of the strut 208^{II}, being the longest, and each subsequent segment 212^{II}b, and 212^{II}c having a progressively shorter length. For example, strut 208^{II} can have a total length L^{II}, and the segments 212^{II}a, 212^{II}b, and 212^{II}c can have corresponding lengths L^{II}a, L^{II}b, and L^{II}c. In the example illustrated in FIG. 4B, L^{II}a>L^{II}b>L^{II}c.

In some examples, struts 208^{I} are the first-type struts, and struts 208^{II} are the second-type struts. In some examples, the length of the proximal-most segment 212^{I}a of the first-type strut 208^{I} can be longer than the length of any of the segments 212^{II} of the second-type strut 208^{II}, such that L^{I}a>{L^{II}a, L^{II}b, L^{II}c}. In some examples, the lengths of the segments 212^{II}b, and 212^{II}c can be equal to the lengths of the segments 212^{I}a and 212^{I}b, respectively, such that L^{I}b=L^{II}a and L^{I}c=L^{II}b. In some examples, the length of the distal most segment 212^{II}c of the second-type strut 208^{II} can be shorter than the length of any of the segments 212^{I} of the first-type strut 208^{I}, such that L^{II}c<{L^{I}a, L^{I}b, L^{I}c}. In some examples, the first-type strut 208^{I} is longer than the second-type strut 208^{II}, such that L^{I}>L^{II}.

In the illustrated examples, each segment 212 of the strut 208 (including any first-type strut and/or second-type strut) is curved such that the overall shape of the entire strut 208 is curved with respect to the lateral axis 12 (or any line parallel to axis 12 and perpendicular to axis 10) within the plane 20. As used in the present specification, a component, such as a strut or strut segment, being curved with respect to a particular axis, means that the component curves around that axis and that axis is parallel to a line that is perpendicular to plane 20 and extends through the center of curvature of the curve. In other words, the strut 208 can be thought of as a straight bar that has been bent around axis 12 (which extends into and out of the plane 20) to form a curve. Lateral axis 12 is parallel to a line that extends through the center of curvature of the strut 208.

The term “entire strut” refers to the strut including all of its segments, as opposed to a “portion of the strut” that may refer to only some of the segments, such as only a portion that includes the segments subsequent to the proximal-most segment. It is to be understood that any type of strut 208 disclosed herein, refers to a unitary strut, meaning that no segment 212 of a strut 208 is pivotable or rotatable relative to any other segment 212 of the same unitary strut 208. In some examples, a unitary strut 208 can be formed from a unitary piece of material. For example, a unitary strut 208 can be formed by processing and machining procedures such as laser cutting, waterjet cutting, etc.

In some examples, each strut can have a continuous and constant curve from one end of the strut to the other end of the strut. In other examples, the projection of each segment 212 in a plane parallel to the longitudinal axis 10 can be straight (i.e., each segment 212 is straight except for any helical curvature with respect to the longitudinal axis 10) and the amount of offset of each segment 212 relative to an adjacent segment 212 along the length of strut 208 can vary such that the overall shape of the strut 208 is curved along its length with respect to the lateral axis 12 (or any line parallel to axis 12 and perpendicular to axis 10); that is, a line extending from one end of the strut to the other end and intersecting each segment 212 is curved with respect to axis 12. Alternatively, individual strut segments 212 can be straight and connected end-to-end to each other at non-zero angles such that the overall shape of the strut 208 is curved along its length with respect to the lateral axis 12 (or any line parallel to axis 12 and perpendicular to axis 10).

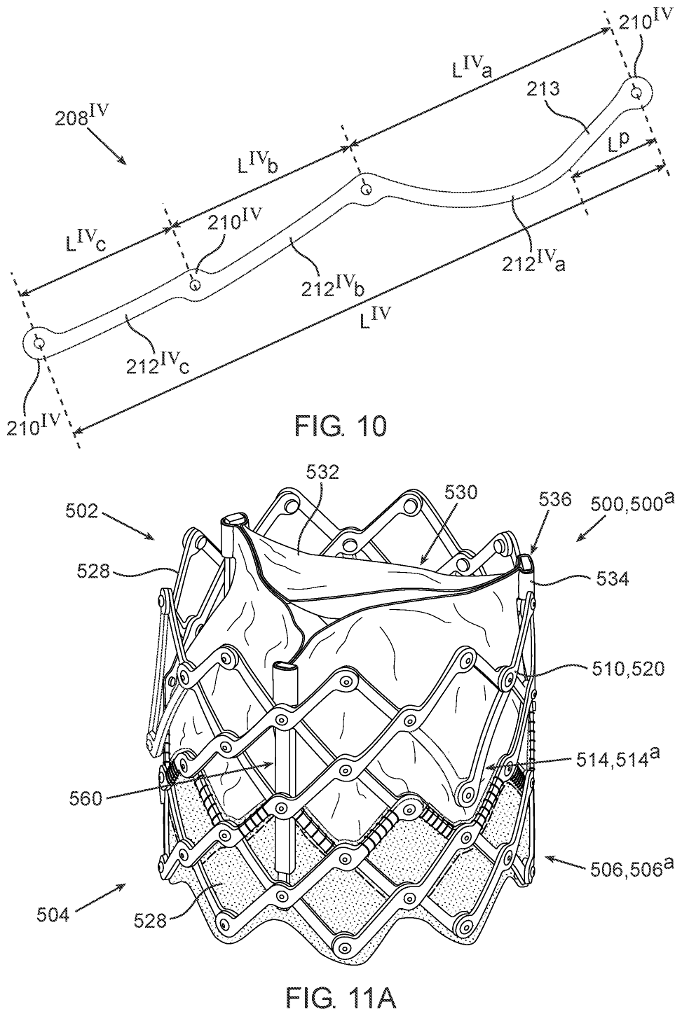

In other examples, one or more of the struts of a frame can have a non-constant or variable curvature along its length (in which case the center of curvature of the strut can vary as one moves along the length of the strut). For example, the radius of curvature can be greater along segment 212^{I}a and smaller along segments 212^{I}b and 212^{a}c. Another example of a segment 212^{IV}a having a radius of curvature significantly larger than that of other segments of the same strut 208^{IV} are shown and will be described in conjunction with FIG. 10 further below.

The frame 206 comprises a plurality of rows of cells, that include at least a first, uppermost, row of cells extending from the outflow end 202, and a second row of cells, wherein the number of cells in the second row is higher than the number of cells in the first row. In the assembled frame 206^{a}, formed by interconnected first-type and second-type struts 208^{I} and 208^{II}, three rows of cells are defined, including a first row of cells 214, a second row of cells 216, and a third row of cells 218 with the cells 214 being the largest, the cells 216 being smaller than the cells 214 and the cells 218 being smaller than the cells 216. The cells 214, which can be the proximal most cells of the valve 200, are also termed the commissure cells 214 as they are configured to support commissures 236 of the leaflet assembly 230 that can be attached thereto, while cells 216 and 218 can be also termed non-commissure cells.

It is to be understood that the term “cell” refers to a closed cell, which is defined by four segments 212 intersecting at at-least three junctions 220. All of the closed cells of the frame 206^{a}, for example, are defined by four segments 212 extending between four junctions 220.

As shown in FIG. 3A, each strut 208 (including first-type strut 208^{I} and second-type struts 208^{II}) can be curved and arranged such that it is concave with respect to the outflow end 202. Due to the unique shape of the struts 208, the frame 206 formed by the struts can have a non-Euclidian geometry, and in particular, a hyperbolic geometry (also referred to as Lobachevsky geometry). The frame 206 in the illustrated example therefore can be referred to as a “Lobachevsky” frame. The degree of curvature of a strut 208 in the plane 20 can be defined as the reciprocal of the radius of a circle comprising the strut as an arc, as shown in the following equation:

$${K}_{S} = \frac{1}{R} ; \qquad Equation 1$$

where Kₛ=the curvature of the strut, and R=the radius of a circle comprising the strut as an arc of the circle. In the illustrated example, each strut 208 of frame 206^{a} has the same degree of curvature in the plane 20. However, in other examples, each strut 208 can have a differing degree of curvature in the plane 20. In some examples, due to the elasticity of the struts and the connections between overlapping struts, the degree of curvature of a strut can change during radial expansion and compression of the frame. For example, in a first configuration shown in FIG. 3A, each strut 208 can be deformed such that it has a lesser degree of curvature (each strut is straighter or straight in the plane 20) than when further expanded to a higher degree of expansion shown in FIG. 3B.

The curvature of the struts 208 in plane 20 can give the frame 206 a non-cylindrical, tapered shape (e.g., a frustoconical shape, a V-shape, or a Y-shape) wherein the outflow end 202 has a first diameter D1 larger than a second diameter D2 of the inflow end 204 as shown in FIG. 3A. The degree of taper can be referred to as the draft angle of the frame 206, which can be a measure of the angle between the longitudinal axis 10 and a line 14 drawn tangent to the outer surface of the frame. When implanted (e.g., surgically implanted) within the native annulus of a patient, the larger outflow relative to the inflow created by the tapered shape can reduce the pressure gradient across the prosthetic valve, helping to improve hemodynamics and mitigate the risk of paravalvular leakage. In some examples, the draft angle increases as the valve is expanded to a larger first diameter D1.

In some examples, the draft angle between longitudinal axis 10 and tangent line 14 can be at least 2 degrees, at least 5 degrees, at least 10 degrees, at least 20 degrees, at least 30 degrees, at least 40 degrees, or at least 50 degrees. Each possibility represents a different example. In some examples, the draft angle can be between 2 and 15 degrees. In some examples, the ratio of the outflow diameter D1 to the inflow diameter D2 is at least greater than 1, at least greater than 1.1, at least greater than 1.2, at least greater than 1.3, at least greater than 1.4, or at least greater than 1.5. Each possibility represents a different example.

The varying lengths of the strut segments form angles between pivotably connected struts which progressively decrease from the inflow end 204 to the outflow end 202. For example, angle 238^{a} is shown to be larger than angle 240^{a} in FIGS. 3A-B.

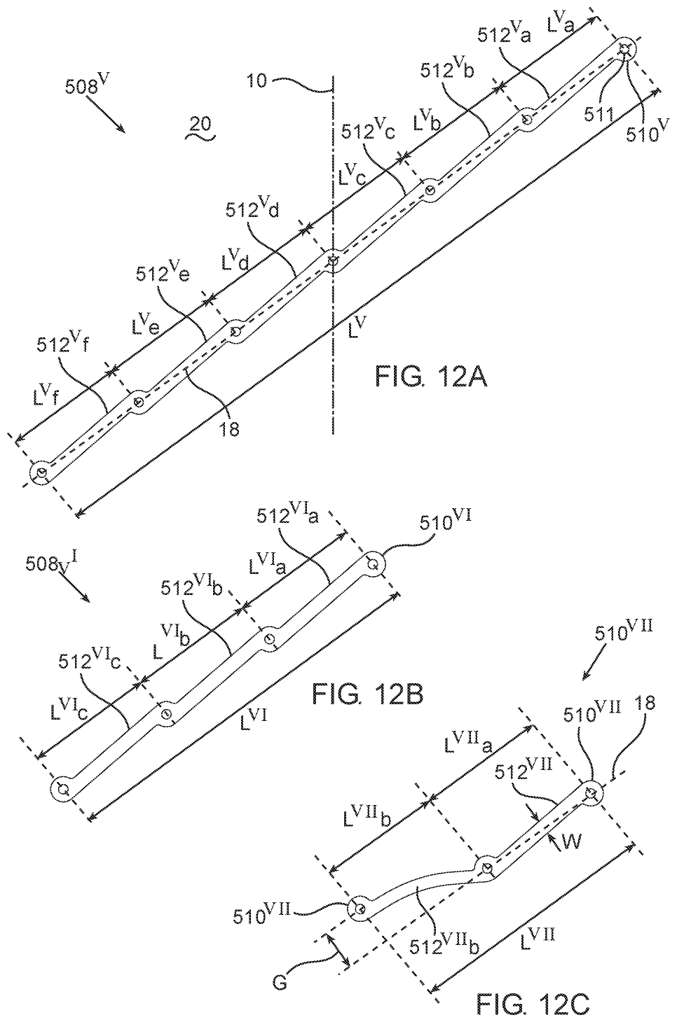

In some examples, one or more segments can have unequal lengths and one or more segments can have equal lengths. For example, the segment 212¹a can be the longest segment, while segments 212^{II}b and 212^{II}c can have equal lengths, such that L^{II}a>L^{II}b and L^{II}b=L^{II}c. In still other examples, the connection portions 210 can be equally spaced along the length of each strut, forming segments of equal lengths, such that L^{II}a=L^{II}b=L^{II}c, such as shown for equal-length segments 512^{V} or 512^{VI} of strut examples 508^{V} or 508^{vI} illustrated in FIGS. 12A and 12B, respectively. Similarly, in some examples L^{I}a>L^{I}b and L^{I}b=L^{I}c, or alternatively L^{I}a=L^{I}b=L^{I}c.

As shown in FIG. 3A, each strut 208 can be curved helically with respect to the longitudinal axis 10 of the frame to define an annular shape of the frame 206. The helical curve provides each strut 208 with a concave, radial inner surface (the surface facing the longitudinal axis 10) and an opposing convex, radial outer surface (the surface facing away from the longitudinal axis 10).

In some examples, as shown in FIGS. 3A-B, none of the struts 208 of frame 206^{a} fully extends all the way from the inflow end 204^{a} to the outflow end 202^{a}. The first-type struts 208^{I} extend from outflow apices 224 at the outflow end 202 to non-apical junctions 222 that are proximal to the inflow end 204, such as distal-most non-apical junctions 222b. Thus, frame 206^{a} comprises pairs of first-type struts 208¹, and more specifically, proximal-most segments 212^{I}a of first-type struts 208^{I}, that intersect at corresponding outflow apices 224. The second-type struts 208^{II} extend from inflow apices 226 at the inflow end 204 and terminate at non-apical junctions 222 that are distal to the outflow end 202, such as proximal-most non-apical junctions 222a, as illustrated. Thus, frame 206^{a} comprises pairs of second-type struts 208^{b} that intersect at corresponding inflow apices 226.

This arrangement results in the second row of cells 216 including the maximal number of cells, such as six cells 216^{a} in the illustrated examples, while each of the first row of cells 214^{a} and the third row of cells 216^{a} includes half as much, i.e., three cells in each respective row. In effect, the first row includes only commissure cells 214, and no non-commissures cells.

The three commissure cells 214 may correspond to three commissures 236 that can be attached thereto, in tricuspid arrangements of the leaflet assembly 230. The reduced number of cells at the first row of cells 216 and at the third row of cells 218 advantageously removes some of the constraints imposed on pivotable movement of the struts 208 during valve expansion, thereby increasing the range of expansion diameters that prosthetic valve 200 can assume. For example, FIG. 3B shows the frame 206^{a} further expanded with respect to the configuration shown in FIG. 3A to a larger outflow diameter D1, during which the angle 238 is substantially increased to a degree that is much higher than that possible for a conventional frame in which all struts would have spanned the entire length between the inflow end 204 and the outflow end 202. In some implementations, the angle 238 may assume a value that is higher than 180 degrees, meaning that the segments 212^{II} extending from the corresponding inflow end 204 can be flipped toward the opposite junction of the cell 216, in the absence of other limiting mechanisms such as a locking mechanism of an expansion and locking assembly, as will be further elaborated hereinbelow.

The arrangement also results in the frame 206 including proximal-most non-apical junctions 222a from which exactly three strut segments 212 extend at different directions, opposed to conventional frames in which all non-apical junctions usually have four strut segment extending therefrom. In the specific example illustrated in FIGS. 3A-B, for a valve 200^{a} that includes second-type struts 208^{II} with three segments 212^{II}, the frame 206^{a} also includes distal-most non-apical junctions 222b from which exactly three segments 212 extend at different directions.

The varying lengths of the segments 212 also result in varying heights of the cells of each row, wherein the height of each cell is defined as the distance between its axially opposing junctions 220. In some examples, as shown in FIGS. 3A-B, the cells in each row have a progressively increasing height between the inflow end 204 and the outflow end. In some examples, the height of the first row of cells 214 is greater than the height of the second row of cells 216. In further examples, the height of the second row of cells 216 is also greater than the height of the third row of cells 218.

According to some examples, as shown in FIG. 2, mechanically expandable surgically implantable prosthetic valve 200^{a} further comprises a plurality of support members 242 that can be made of a relatively flexible and soft material, including synthetic materials (e.g., PET fabric) or natural tissue (e.g. bovine pericardium), attached to the segments 212 of commissure cells 214^{a}. The number of support members 242 can match the number of commissures 236, wherein each commissure 236 can be mounted to the frame 206 by being attached (e.g., sutured) to a corresponding support member 242.

In some examples, each support member 242 is attached (e.g., sutured) to each of the four segments 212 forming a commissure cell 214^{a} of the frame 206^{a}. In some examples, the support member 242 covers the entire area of the corresponding commissure cell 214^{a}.

A commissure 236 can be formed by folding the tabs 234 and stitching them to each other, and/or to additional components of the commissure, such as reinforcement members, fabrics and the like, according to various configurations disclosed in US Pat. Publication No. 2018/0028310, which is incorporated herein by reference. The commissure 236 can be then attached to the respective support member 242, for example by suturing it to the support member 242 along a portion that may extend at least partially between the outflow apex 224 and the opposite lower non-apical junction 222 of the corresponding commissure cell 214^{a}.

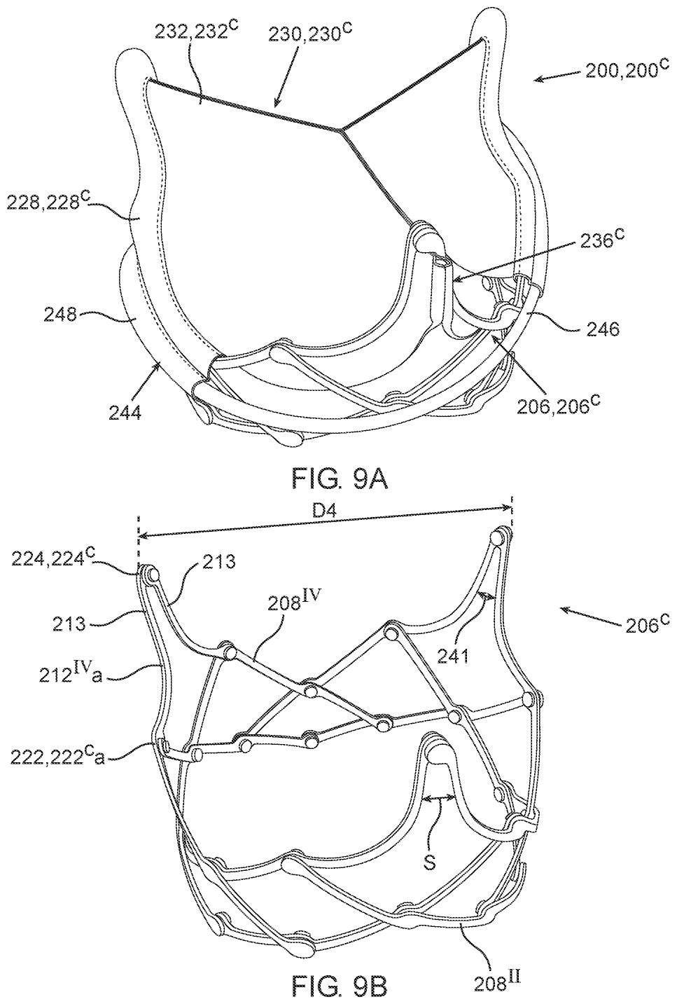

According to some examples, the mechanically-expandable surgically implantable prosthetic valve 200 further comprises a soft sealing or sewing ring 244 that circumscribes the frame 206, for example around the skirt 228, and may be used to secure the valve 200 to a native annulus such as with sutures. The sewing ring 244 comprises a sewing ring insert 246 and a cloth cover 248 around the ring insert 246 (hidden from view in FIG. 2, shown for example in FIG. 9A). The sewing ring insert 246 can be made of a suture permeable material for suturing the valve to a native annulus, as known in the art. For example, the sewing ring insert 246 can be made of a silicone-based material, although other suture-permeable materials can be used.

The cloth cover 248 can be formed of any biocompatible fabric, such as, for example, polyethylene terephthalate or polyester fabric. The sewing ring 244 can be secured to the frame 206 by being stitched to the skirt 238, or via sutures extending through the sewing ring 244 and stitched directly around struts 208. In some examples, the sewing ring 244 can be disposed between the proximal-most non-apical junctions 222a and the distal-most non-apical junctions 222b.

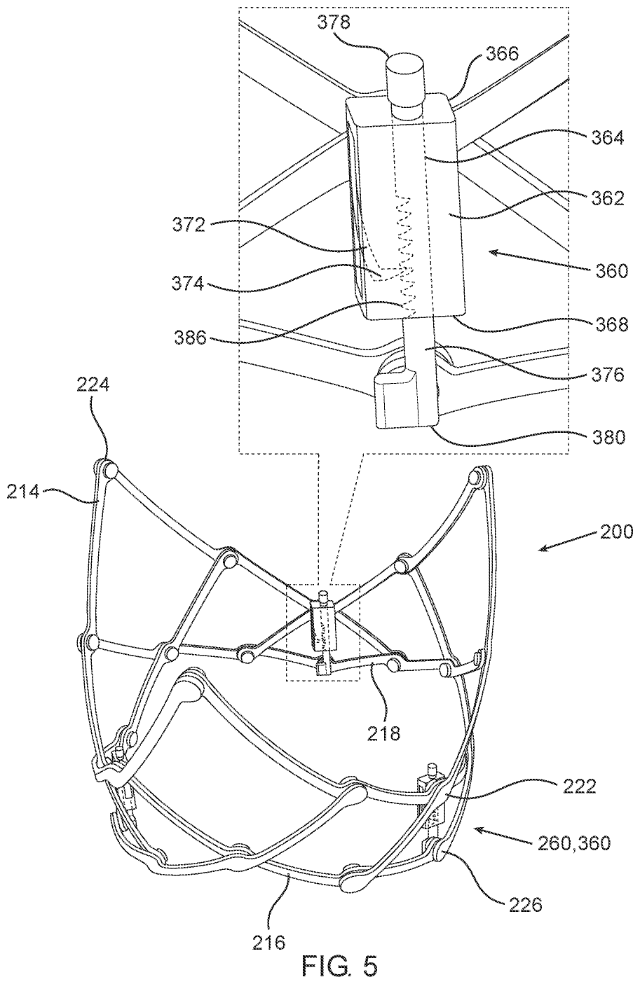

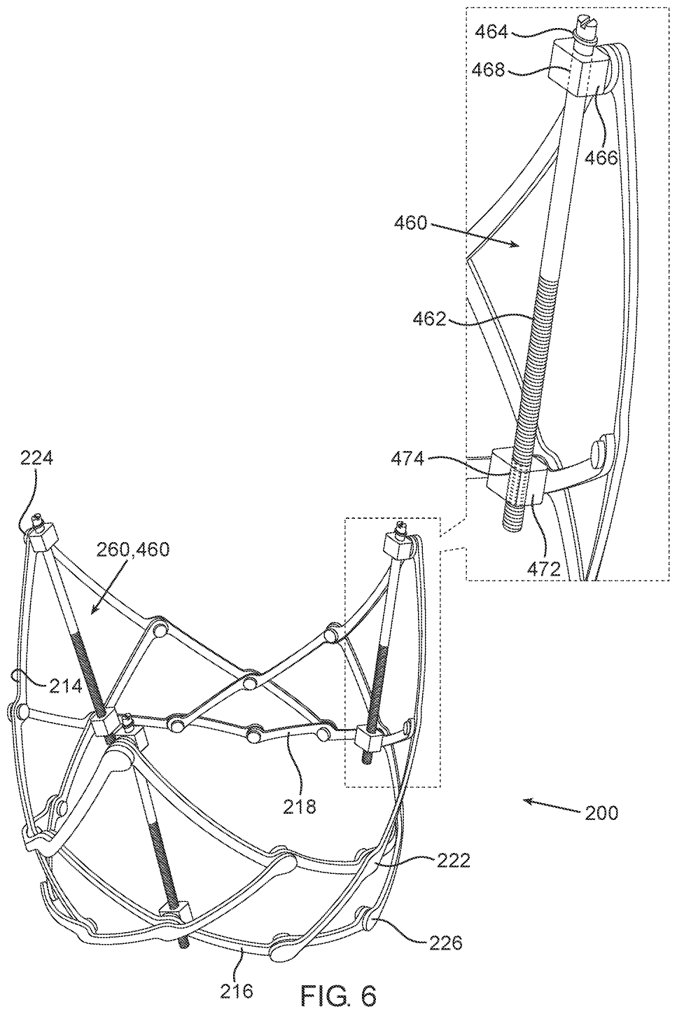

According to some examples, a mechanically-expandable surgically implantable prosthetic valve 200 comprises a plurality of expansion and locking assemblies 260, configured to facilitate expansion or contraction of the valve 200, and in some instances, to lock the valve 200 at selected expanded state, preventing unintentional compression thereof. FIG. 5 shows a prosthetic valve 200 equipped with one type of expansion and locking assemblies 360, implemented with a ratcheting mechanism, and FIG. 6 shows a prosthetic valve 200 equipped with another type of expansion and locking assemblies 460, implemented with threaded mechanism. Although each of FIGS. 5 and 6 illustrates three expansion and locking assemblies 260, mounted to the frame 206, and optionally equiangularly spaced from each other around an inner surface thereof, it should be clear that a different number of expansion and locking assemblies 260 may be utilized, that the expansion and locking assemblies 260 can be mounted to the frame around its outer surface, and that the circumferential spacing between expansion and locking assemblies 260 can be unequal.

The ratcheting-type expansion and locking assembly 360 shown in FIG. 5 may include an outer member 362 defining an outer member lumen 364, secured to a component of the valve 200, such as the frame 206, at a first location, and an inner member 376 secured to a component of the valve 200, such as the frame 206, at a second location, axially spaced from the first location.

The inner member 376 extends between an inner member proximal end portion 378 and an inner member distal end portion 380. The inner member 376 comprises an inner member coupling extension 384 (hidden from view in FIG. 5) extending from its distal end portion 380, which may be formed as a pin extending radially outward from the distal end portion 380, configured to be received within respective apertures 211 of struts 208 intersecting at a junction 220. The inner member 376 may further comprise a linear rack having a plurality of ratcheting teeth 386 along at least a portion of its length. According to some examples, inner member 376 further comprises a plurality of ratcheting teeth 386 along a portion of its outer surface.

The outer member 362 comprises an outer member proximal end portion 366 defining a proximal opening of its lumen 364, and an outer member distal end portion 368 defining a distal opening of its lumen 364. The outer member 362 can further comprise an outer member coupling extension 370 (hidden from view in FIG. 5) extending from its proximal end portion 366, which may be formed as a pin extending radially outward from the external surface of the proximal end portion 366, configured to be received within respective apertures 211 of struts 208 intersecting at a junction 220.

The outer member 362 can further comprise a spring-biased arm 372, attached to or extending from one sidewall of the outer member 362, and having a tooth or pawl 374 at its opposite end, biased inward toward the inner member 376 when disposed within the outer member lumen 364.

At least one of the inner or outer member 376 or 362, respectively, is axially movable relative to its counterpart. The expansion and locking assembly 360 in the illustrated example, comprises a ratchet mechanism or a ratchet assembly, wherein the pawl 374 is configured to engage with the teeth 386 of the inner member 376. The spring-biased arm 372 can comprise an elongate body terminating in a pawl 374 in the form of a locking tooth, configured to engage the ratcheting teeth 386 of the inner member 376. The pawl 374 can have a shape that is complimentary to the shape of the teeth 386, such that the pawl 374 allows sliding movement of the inner member 376 in a first direction relative to the spring-biased arm 372 (e.g., a proximally oriented direction) and resists sliding movement of the inner member 376 in the opposite second direction (e.g., a distally oriented direction) when the pawl 374 is in engagement with one of the teeth 386.

The arm 372 can be biased inwardly such that the pawl 374 is resiliently retained in a position engaging one of the teeth 386 of the inner member 376. In the illustrated example, the spring-biased arm 372 is configured as a leaf spring. In some examples, the spring-biased arm 372 can be integrally formed with the outer member 362, in other examples, the spring-biased arm 372 can be separately formed and subsequently coupled to the outer member 362. The biased configuration of the arm 372 ensures that under normal operation, the pawl 374 stays engaged with the teeth 386 of the inner member 376.

The spring-biased arm 372 can be formed of a flexible or resilient portion of the outer member 362 that extends over and contacts, via its pawl 374, an opposing side of the outer surface of the inner member 376. According to some examples, the spring-biased arm 372 can be in the form of a leaf spring that can be integrally formed with the outer member 362 or separately formed and subsequently connected to the outer member 362. The spring-biased arm 372 is configured to apply a biasing force against the outer surface of the inner member 376, so as to ensure that under normal operation, the pawl 374 stays engaged with the ratcheting teeth 386 of the inner member 376.

In the illustrated example shown in FIG. 5, the first location to which the outer member 362 is attached can be a proximal non-apical junction 222 of a cell 218 along the third row, and the second location to which the inner member 376 is attached can be an inflow apex 226 of the same cell 218. In alternative configurations, the first location to which the outer member 362 is attached can be an outflow apex 224, and the second location to which the inner member 376 is attached can be a distal non-apical junction 222 of the same commissure cell 214, similar to the attachment locations shown in FIG. 6. In yet alternative configurations, the first and second locations can be opposite non-apical junctions 222, such as a proximal-most non-apical junction 222a and a distal-most non-apical junction 222b of a cell 216 of the second row.

It is to be understood that while the illustrated examples are for expansion and locking assemblies secured to a proximal junction serving as the first location, and to a distal junction serving as the second location, in other implementations, the expansion and locking assembly can be secured to other junctions. For example, any expansion and locking assembly can be secured to a distal junction 220 (which can be either an inflow apex 226 or a non-apical junction 222) via the outer member coupling extension 370, serving as the first location, and to an opposing proximal junction 220 (which can be either an outflow apex 224 or a non-apical junction 222) axially aligned therewith, via the inner member coupling extension 384, serving as the second location.

Actuating means (not shown) can releasably engage components of the expansion and locking assemblies 360, such as inner members 376, such that when actuating means pull the inner members 376, preferably while providing a counter-force to hold the outer members 362 in place, the inflow end 204 and the outflow end 202 are approximated, thereby facilitating valve expansion. It is to be understood that the frame 206 can expand radially outward by either axially moving the inner member 376 upward (i.e., in a proximally oriented direction), relative to the outer member 362, and/or by axially pushing an outer member 362 downward (i.e., in a distally oriented direction), relative to an inner member 376.

In the example illustrated in FIG. 5, the inner member coupling extension 384 extends through apertures 211 in two struts 208 interconnected at an inflow apex 226, while the outer member coupling extension 370 extends through aperture 211 in two struts 208 interconnected at a proximal non-apical junction 222 of the same cell 218. As such, when the inner member 376 is moved axially, for example upward (or in a proximally oriented direction), within the outer member lumen 364, the inner member coupling extension 384 moves along with the inner member 376, thereby causing the portion to which the inner member coupling extension 384 is attached to move axially as well, which in turn causes the frame 206 to foreshorten axially and expand radially.

The struts 208 to which the inner member coupling extension 384 is connected are free to pivot relative to the coupling extension 384 and to one another as the frame 206 assumes various expansion diameters. In this manner, the inner member coupling extension 384 serves as a fastener that forms a pivotable connection between those struts 208. Similarly, struts 208 to which the outer member coupling extension 370 is connected are also free to pivot relative to the coupling extension 370 and to one another as the frame 206 assumes various expansion diameters. In this manner, the outer coupling extension 370 also serves as a fastener that forms a pivotable connection between those struts 208.

As mentioned above, when the pawl 374 of the spring-biased arm 372 is engaged with the ratcheting teeth 386, the inner member 376 can move in one axial direction, such as the upward or proximally oriented direction, but cannot move in the opposite axial direction. This ensures that while the pawl 374 is engaged with the ratcheting teeth 386, the frame 206 can be further radially expanded but cannot be radially compressed. In this manner, the actuation mechanism also serves as a locking mechanism of the prosthetic valve 200.

When the valve 200 is surgically implanted within the patient's site of implantation, such as being sutured via its sewing ring 244 to the native aortic annulus, the native annulus may exert radial forces against the prosthetic valve 200 that would strive to compress it. However, the engagement between the pawl 374 of the spring-biased arm 372 and the ratcheting teeth 386 of the inner member 376 prevents such forces from compressing the frame 206, thereby ensuring that the frame 206 remains locked in the desired expanded diameter.

The alternative threaded-type expansion and locking assembly 460 shown in FIG. 6 may include a screw or threaded rod 462, a first anchor 466 in the form of a cylinder or sleeve defining a first anchor channel 468, secured to a component of the valve 200, such as the frame 206, at a first location, and a second anchor 472 in the form of a threaded nut defining second anchor threaded channel 474, secured to a component of the valve 200, such as the frame 206, at a second location, axially spaced from the first location. The rod 462 extends through the first anchor channel 468 and the second anchor threaded channel 474.

The first anchor 466 comprises a first anchor coupling extension 470 (hidden from view in FIG. 6), which may be formed as a pin extending radially outward therefrom, configured to be received within respective apertures 211 of struts 208 intersecting at a junction 220 at the first location. The second anchor 472 may similarly comprise a second anchor coupling extension 476 (hidden from view in FIG. 6), which may be formed as a pin extending radially outward therefrom, configured to be similarly received within respective apertures 211 of struts 208 intersecting at a junction 220 at the second location.

While the first and second locations are illustrated in FIG. 6 at opposite ends of commissure cells 214, any other attachment configuration described hereinabove in relation to the ratcheting-type expansion and locking assembly 360 may be similarly applied to the threaded-type expansion and locking assembly 460, such that each expansion and locking assembly 460 is configured to either increase the distance between the attachment locations of respective first and second anchors 466 and 472, which causes the frame 206 to elongate axially and compress radially, or to decrease the distance between the attachment locations of a respective first and second anchors 466 and 472, which causes the frame 206 to foreshorten axially and expand radially.

For example, each rod 462 can have external threads that engage internal threads of the second anchor threaded channel 474 such that rotation of the rod 462 causes corresponding axial movement of the second anchor 472 toward or away from the first anchor 466 (depending on the direction of rotation of the rod 462). This causes the first and second locations supporting the first anchor 466 and the second anchor 472 to move closer towards each other to radially expand the frame 206 or to move farther away from each other to radially compress the frame 206, depending on the direction of rotation of the rod 462.

In yet further examples, the expansion and locking assemblies can be reciprocating type assemblies configured to apply axial directed forces to the frame 206 to produce radial expansion and compression of the frame 206. For example, the rod 462 of each expansion and locking assembly 460 can be fixed axially relative to the second anchor 472 and slidable relative to the first anchor 466. Thus, in this manner, moving the rod 462 downward or distally relative to the first anchor 466 and/or moving the first anchor 466 upward or proximally relative to the rod 462 radially compresses the frame 206. Conversely, moving the rod 462 upward or proximally relative to the first anchor 466 and/or moving the first anchor 466 downward or distally relative to the rod 462 radially expands the frame 206.

The above-mentioned reciprocating type assemblies can be utilized for expansion (or compression) of the frame 206, and the prosthetic valve 200 can also include one or more locking mechanisms, configured to retain the frame 206 in the desired expanded diameter. The locking mechanisms can be separate components that are mounted on the frame apart from the expansion assemblies, or they can be a sub-component of the expansion assemblies themselves. In particular examples, the assemblies can comprise combination expansion and locking mechanism, as further described in U.S. Publication No. 2018/0153689, which is incorporated herein by reference.

Each rod 462 can include an engagement head 464 configured to receive or engage with external drivers (not shown). For example, the engagement head 464 can be provided in the form of a slot or otherwise formed screw-head, configured to engage a screw-driver that may apply rotational movement to the rod.

In some examples, the internal friction or resistance of the threaded rod 462 within the second anchor threaded channel 474 can be sufficient to retain the frame 206 in a desired expanded diameter.

While specific expansion and locking mechanisms are described above, utilizing a ratcheting mechanism between inner and outer members of the expansion and locking assemblies 360, or screw-engagement mechanisms between a bolt-like threaded rod and a nut-like second anchor, other mechanisms may be employed to promote relative movement between members of expansion and locking assemblies 260. Further details regarding the structure and operation of expansion and locking assemblies for mechanically expandable valves are described in U.S. Pat. Nos. 9,827,093, 10,603,165, and 10,806,573, U.S. Patent Application Publication No. 2018/0344456, and US Patent Application Nos. 62/870,372 and 62/776,348, all of which are incorporated herein by reference.

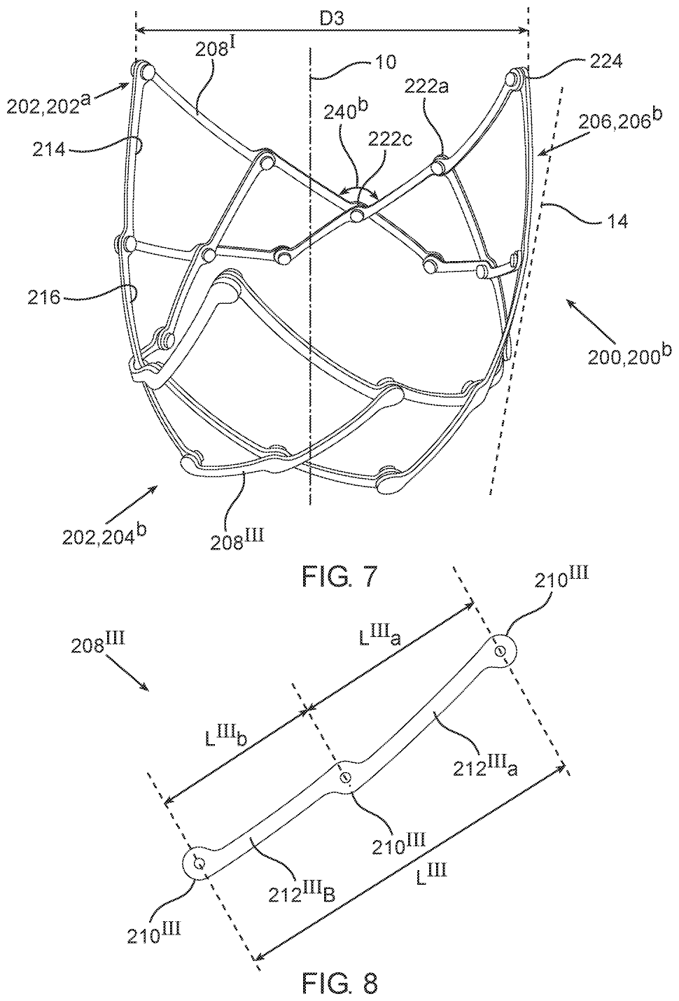

FIG. 7 shows another example of a mechanically-expandable surgically-implantable prosthetic valve 200^{b}, which is generally similar to the prosthetic valve 200^{a}, except that the frame 206^{b} includes only a first row of cells 214 and a second row of cells 216, without a third row of cells. Such a frame 206^{b} can be formed by interconnecting first-type struts 208^{I} with another type of second-type struts 208^{III}, as will be further described hereinbelow.

FIG. 8 shows a flattened projection of an example of a strut 208^{III}, comprising two segments 212^{III}a and 212^{III}b, with segment 212^{III} a, which can be the proximal segment of the strut 208^{III}, being longer than distal segment 212^{III}b. For example, strut 208^{III} can have a total length L^{III}, and the segments 212^{III}a and 212^{III}b can have corresponding lengths L^{III}a and L^{II}b. In the example illustrated in FIG. 8, L^{III}a>L¹b. In some examples, the lengths of the strut segments 212^{III}a, and 212^{III}b can be equal to the lengths of the strut segments 212^{I}b and 212^{I}c, respectively, such that L^{I}b=L^{III}a and L^{I}c=L^{II}b. In some examples, strut 208^{III} can be identical to a second-type strut 208^{II} except that it is devoid of a third segment 212¹c.

In some examples, struts 208^{I} are the first-type struts, and struts 208^{III} are the second-type struts. In the assembled frame 206^{b} shown in FIG. 7, the first-type struts 208^{I} fully extend all the way from the inflow end 204^{b} to the outflow end 202^{b}, and more specifically, from outflow apices 224 to inflow apices 226. Thus, frame 206^{b} comprises pairs of first-type struts 208^{I}, and more specifically, proximal-most segments 212^{I}a of first-type struts 208^{I}, that intersect at corresponding outflow apices 224, as well distal-most segments 212^{I}c of pairs of first-type struts 208^{I} that intersect at corresponding inflow apices 226.

The second-type struts 208^{III}, in contrast, do not fully extend all the way from the inflow end 204^{b} to the outflow end 202^{b}, but rather extend from inflow apices 226 and terminate at non-apical junctions 222 that are distal to the outflow end 202, such as proximal-most non-apical junctions 222a, as illustrated. Thus, frame 206^{b} comprises pairs of second-type struts 208^{III} that intersect at the remainder of inflow apices 226 that are not defined by intersecting pairs of first-type struts 208^{I}.

Similar to the case with frame 206^{a}, this arrangement results in the second row of cells 216 including the maximal number of cells, such as six cells 216^{b} in the illustrated example, while the first row of cells 214^{b} includes half as much, i.e., three commissure cells 214^{b}, and no non-commissures cells. However, unlike frame 206^{a}, the arrangement of the struts of frame 206^{b} also results in a plurality of mid non-apical junctions 222c disposed equiangularly around the second row, that are not bound by any closed cells either from above (i.e., proximal to) or below (i.e., distal to) the mid non-apical junctions 222c. This in turn significantly increases the extent to which the frame can be expanded, due to the increased degree of freedom of the pivot points at these mid non-apical junctions 222c, resulting in a frame 206^{b} that can be expanded to a maximal outflow diameter D3 that may be significantly larger than D2 shown for a frame 206^{a}, and defining a significantly larger draft angle between the longitudinal axis 10 and the tangent line 14 of the frame 206^{b}, compared to a frame 206^{a} including similarly dimensioned first-type struts 208^{I}.

While both frames 206^{a} and 206^{b} can include first-type struts 208^{I} of the same length and shape, the different types of second-type struts interconnected therewith can result in the frame 206^{b} shown in FIG. 7 being able to expand to a larger maximal outflow diameter D3, with respect to the maximal outflow diameter D2 of the frame 206^{a}, forming a maximal angle 240^{b} that can be similarly larger for the frame 206^{b} than the maximal angle 240^{a} of the frame 206^{a}.

FIGS. 9A-B show perspective views of another example of a mechanically-expandable surgically-implantable prosthetic valve 2000, shown with (FIG. 9A) and without (FIG. 9B) soft components (such as a skirt, a sewing ring and a leaflet assembly). Prosthetic valve 2000 can be generally similar to the prosthetic valve 200^{a}, except that the frame 2060 can be formed by interconnecting another a different type of first-type struts 208^{IV} with second-type struts 208^{II} (see FIGS. 9A-B) of 208^{III}, as will be further described below.

FIG. 10 shows a flattened projection of an example of a strut 208^{IV}, comprising three segments 212^{IV}a, 212^{IV}b, and 212^{IV}c, with segment 212^{IV}a, which can be the proximal-most segment of the strut 208^{IV}, being the longest, and having a larger, optionally varying, curvature, compared to the curvature of any of the subsequent segments 212^{IV}b and 212^{IV}c. In some examples, strut 208^{IV} can be identical to strut 208^{I} except that the proximal-most segment 212^{IV}a has a larger radius of curvature than any of the subsequent segments (such as segments 212^{IV}b and 212^{IV}c), and a radius of curvature that is larger than that of the proximal-most segment 212^{I}a of the example of strut 208^{I} shown in FIG. 4A.

In some examples, struts 208^{IV} are the first-type struts, and struts 208^{II} are the second-type struts (see FIGS. 9A-B). In some examples, struts 208^{IV} are the first-type struts, and struts 208^{III} are the second-type struts (not shown). As shown in FIGS. 9A-B, the proximal-most segments 212^{IV}a are curved so as to define proximal portions 213 of the proximal-most segments 212^{IV}a, shaped such that the proximal portions 213 of each pair of first-type struts 208^{IV} of a corresponding commissure cells 214^{c} are substantially parallel to each other along the functional range of outflow diameters D4 of the valve 200^{c}. According to some examples, each commissure 236^{b} is attached to a pair of proximal portions 213.

According to some examples, each pair of adjacent proximal portions 213 define a commissure angle 241 formed there-between at the outflow end 202^{c}. In some examples, the commissure angle can change as the valve is expanded or compressed, but does not exceed a maximal threshold that is lower than 30 degrees at the maximal diameter of the prosthetic valve 200^{c}, which is defined as the maximal outflow diameter D4 within the functional range of diameters the valve can assume for the process of implantation. In some examples, the commissure angle can be zero at least at one outflow diameter D4, meaning that both proximal portions 213 are completely parallel to each other at this diameter. In some examples, the commissure angle 241 is not greater than 5 degrees at the maximal diameter of the prosthetic valve 200^{c}. In some examples, the commissure angle 241 is not greater than 10 degrees at the maximal diameter of the prosthetic valve 200^{c}. In some examples, the commissure angle 241 is not greater than 15 degrees at the maximal diameter of the prosthetic valve 200^{c}.

The proximal portion 213 can have a length L^{p}, along which the proximal portion 213 is relatively straight or has the highest radius of curvature, which can be large enough to result in a near-straight configuration thereof. According to some examples, the length of the proximal portion 213 is at least as great as a quarter of the length of the proximal-most segment 212^{IV}a, such that L^{p}≥(0.25*L^{IV}a). According to some examples, the length of the proximal portion 213 is at least as great as a third of the length of the proximal-most segment 212^{IV}a, such that L^{p}≥(0.33*L^{IV}a). According to some examples, the length of the proximal portion 213 is at least as great as half of the length of the proximal-most segment 212^{IV}a, such that L^{p}≥(0.5*L^{IV}a).

As further shown in FIGS. 9A-B, the proposed configuration of the nearly parallel proximal portions 213 of the first-type struts 208^{IV} results in commissure cells 214^{I} that are formed to have a shape which is closer to that of the commissure posts 114 of conventional surgically implantable prosthetic valves 100, allowing the commissures 236^{c} to be formed and attached to the commissure cells 214^{c} in a manner that is similar to that shown and described in conjunction with the attachment of commissures 136 to commissure posts 114.

In some examples, each pair of adjacent proximal portion 213 are attached to each other at their respective connection portions 210^{IV} together forming an outflow apex 224. In some examples, the respective connection portions 210^{IV} of each pair of adjacent proximal portion 213 of the same commissure cell 214^{c} are spaced apart from each other by a distance S such that each outflow apex 224^{c} comprises two connection portions 210^{IV} spaced apart, while both adjacent proximal portions 213 are attached to a corresponding commissure 236 extending there-between (for example, via sutures). Assuming that the proximal portion 213 are substantially parallel to each other, the distance S can be the average distance along their lengths.

According to some examples, the distance S between the connection portions 210^{IV} together forming a single outflow apex 224^{c} is not greater than four times the thickness of the tabs 234 of the leaflets 232. This distance may be adapted to accommodate two leaflets tabs 234, and two layers of a protective cloth (not shown) that may be used to form a commissure 236, assuming that the thickness of the cloth is comparable to the thickness of the tab 234. According to some examples, the distance S between the connection portions 210^{IV} together forming a single outflow apex 224^{c} is not greater than twice the thickness of the tabs 234 of the leaflets 232.

While illustrated in combination with prosthetic valve 200^{a}, it is to be understood that any type of expansion and locking assemblies 260 described hereinabove can be used with other types of mechanically-expandable surgically implantable valves 200, such as valve 200^{b} or 200^{c}. Similarly, while illustrated in combination with second-type struts 208^{II}, it is to be understood that first type struts 208^{IV} can be used with other types of second-type struts, such as struts 208^{III}.

While the mechanically-expandable surgically implantable valves 200 are described above with first-type struts and second type struts having curved segments, it is to be understood that in alternative examples, a frame 206 of a mechanically-expandable surgically implantable valve 200 can comprise first-type struts and second-type struts having linear segments, similar to those that will be described for struts 508^{V} or 508^{VI} below. In such cases, the number and length of the various segments of the first-struts and the second-struts can be similar to that described for struts 208^{I} and/or struts 208^{II}, mutatis mutandis.

An alternative approach to surgically implanting a prosthetic heart valves includes percutaneous minimally-invasive technique, during which a prosthetic valve is configured to be implanted by way of catheterization. For example, the above-mentioned U.S. Pat. Nos. 6,730,118, 9,827,093 and 10,603,165, describe compressible transcatheter prosthetic heart valve that can be percutaneously introduced in a crimped state on a catheter and expanded in the desired position by balloon inflation, by utilization of a self-expanding frame or stent, or by utilization of a mechanical expansion and locking mechanism. Unlike surgically implantable valves, which are conventionally sutured to the surrounding tissue, transcatheter heart valves are expanded against the annulus and are usually retained in position due to the pressure applied thereby against the native anatomy. An important design consideration for such valves is the ability to remain at the treatment location after deployment without becoming dislodged.

FIGS. 11A and 11B show perspective views of a representative example of a prosthetic valve 500 shown with (FIG. 11A) and without (FIG. 11B) soft components (such as a skirt and a leaflet assembly). The prosthetic valve 500 can be a type of a valve deliverable to a patient's target site over a catheter, which is radially expandable and compressible between a radially compressed, or crimped, configuration, and a radially expanded configuration. Thus, a prosthetic valve 500 can be crimped or retained by a delivery apparatus (not shown) in a compressed configuration during delivery, and then expanded to the expanded configuration once the prosthetic valve 500 reaches the implantation site. The expanded configuration may include a range of diameters to which the valve may expand, between the compressed or crimped configuration and a maximal diameter reached at a fully expanded configuration. Thus, a plurality of partially expanded configurations may relate to any expansion diameter between radially compressed or crimped configuration, and maximally expanded configuration. A prosthetic valve 500 of the current disclosure may include any prosthetic valve configured to be mounted within the native aortic valve, the native mitral valve, the native pulmonary valve, and the native tricuspid valve.

FIGS. 11A-B show a specific exemplary example of a mechanically expandable valve 500, illustrated in an expanded state. The prosthetic valve 500 can comprise an inflow end 504 and an outflow end 502. In some instances, the outflow end 502 is the distal end of the prosthetic valve 500, and the inflow end 504 is the proximal end of the prosthetic valve 500. Alternatively, depending for example on the delivery approach of the valve, the outflow end can be the proximal end of the prosthetic valve, and the inflow end can be the distal end of the prosthetic valve.

The valve 500 comprises an annular frame 506 movable between a radially compressed configuration and a radially expanded configuration, and a leaflet assembly 530 mounted within the frame 506. The frame 506 can be made of various suitable materials as described above for frame 206, including plastically-deformable materials such as, but not limited to, stainless steel, a nickel based alloy (e.g., a cobalt-chromium or a nickel-cobalt-chromium alloy such as MP35N alloy), polymers, or combinations thereof.

The leaflet assembly 530 can be similar to any example described hereinabove for leaflet assembly 230, and include a plurality of leaflets 532 that may be joined at their tabs 534 to form commissures 536. The prosthetic valve 500 can also include an outer skirt, which is not shown in FIG. 11A but can be similar to an outer skirt 228, mounted on the outer surface of the frame 506 and configure to function, for example, as a sealing member retained between the frame 506 and the surrounding tissue of the native annulus against which the prosthetic valve is mounted, thereby reducing risk of paravalvular leakage past the prosthetic valve 500.

The prosthetic valve 500 can also include an inner skirt 528 as shown in FIG. 11A, secured to the inner surface of the frame 506, and may be similarly configured to function, for example, as a sealing member to prevent or decrease perivalvular leakage. An inner skirt 528 can further function as an anchoring region for the leaflets 532 to the frame 506, and/or function to protect the leaflets 532 against damage which may be caused by contact with the frame 506, for example during valve crimping or during working cycles of the prosthetic valve 100. Any of the inner skirt 528 and/or outer skirt can be made of various suitable biocompatible materials, such as, but not limited to, various synthetic materials (e.g., PET) or natural tissue (e.g. pericardial tissue). In some examples, the inner skirt 528 comprises a single sheet of material that extends continuously around the inner surface of the frame 506.

In the illustrated example, the struts 508 are pivotably coupled to one another at one or more pivot joints along the length of each strut. For example, in the illustrated configuration, each of the struts 508 can be formed with apertures 511 (see e.g., FIG. 12A) at opposing ends of the strut and apertures 511 spaced along the length of the strut. Respective hinges can be formed at the locations where struts 508 overlap each other via fasteners, such as rivets or pins that extend through the apertures. The hinges can allow the struts 508 to pivot relative to one another as the frame 506 is radially expanded or compressed, such as during assembly or deployment of the prosthetic valve 500.

The pivot joints of interconnected struts 508 form junctions 520 of the valve 500, that can include outflow apices 524 at the outflow end 502 of the valve 500, inflow apices 526 at the inflow end 504 of the valve 500, and other non-apical junctions 522, such as the proximal-most non-apical junctions 522a shown in FIG. 11B.

Each strut 508 can include a plurality of connection portions 510 at which it may be interconnected with another strut to define the junctions 520, the connection portions 510 defining the segments 512 extending there-between. The connection portions 510 can be formed as enlarged (relative to the segments 512) end portions or enlarged intermediate portions. In some examples, struts 508 include a plurality of apertures 511, each aperture 511 comprised within a connection portion, as shown in FIGS. 12A-B for example. In other examples, a strut can include other coupling means, such as integrally formed pins (not shown) extending from corresponding connection portions 510, that may be configured to be received within apertures 511 of struts 508 interconnected therewith. It is to be understood that any type of strut 508 disclosed herein, refers to a unitary strut, meaning that no segment 512 of a strut 508 is pivotable or rotatable relative to any other segment 512 of the same unitary strut 508. In some examples, a unitary strut 508 can be formed from a unitary piece of material. For example, a unitary strut 508 can be formed by processing and machining procedures such as laser cutting, waterjet cutting, etc.

Frame 506 of a mechanically expandable valve 500 comprises at least three types of interconnected struts 508, at least two of which are having a different length, and each type extending along a different portion of the frame 506 between the inflow end 504 and the outflow end 502. The first-type struts of valve 500 extend along the entire length of the frame 206, from the outflow end 502 to the inflow end 504. The second-type struts of valve 500 do not reach the outflow end 502, but rather extend proximally from the inflow end and terminate at non-apical junctions 522 that are distal to the outflow end 502. The third-type struts extend distally from the outflow end 502, wherein pairs of third-type struts are joined together at a clamp apices 516 to form a clamping portions 514 of the valve 500.

Clamp apex 516 may be a specific type of junction 520 defined only by a pair of third-type struts, without any other struts interconnected at the clamp apex 516. The clamp apex 516 is distal to the outflow apices 524. In some examples, the clamp apex 516 is distal to the proximal-most non-apical junctions 522a. In some examples, the clamp apex 516 is proximal to the inflow end 504. It is to be understood that examples of clamp apices 516 being proximal to the inflow end 504 refer to the closed position of the clamping portions 514, as will be defined further below, and a crimped or compressed configuration of the prosthetic valve 500.

FIGS. 12A, 12B and 12C show flattened projections of three types of struts 508 in a plane 20 parallel to the longitudinal axis 10 of the valve 500. FIG. 12A shows a flattened projection of an example of a strut 508^{V} comprising six linear segments 512^{IV}a to 512^{V}f. The strut 508^{V} can have an offset, or zig-zag, pattern defined by the plurality of offset linear segments 512^{V}. Specifically, each linear segment 512^{V} can be slightly laterally offset from an adjacent linear segment 512^{V} in a direction perpendicular to the overall length of strut 508^{V} to provide the zig-zag pattern of the strut. In some examples, all linear segments 512^{V}a have identical lengths, such that L^{V}a=L^{V}b . . . =L^{V}f.

The amount of offset of each linear segment 512^{V} relative to an adjacent linear segment along the length of the strut 508^{V} can be constant such that an axis 18 can pass through the apertures 511 of each connection portions 510^{V} along the entire length of the strut. In alternative examples, the amount of offset between two adjacent linear segments 512^{V} can vary along the length of the strut. For example, the amount of offset between linear segments 512^{V} adjacent the outflow end of the frame can be greater than the amount of offset between linear segments 512^{V} adjacent the inflow end of the frame, or vice versa.

In alternative examples, the struts 508 can have linear segments 512 that are not offset from each other; that is, the struts are substantially rectangular with longitudinal sides of each strut extending continuously from one end of the strut to the opposite end of the strut without offset portions (examples not shown).

FIG. 12B shows a flattened projection of another example of a strut 508^{VI} comprising three linear segments 512^{VI}a, 512^{IV}b and 512^{IV}c. Strut 508^{VI} can be identical to strut 508^{V}, except that it includes fewer linear segment 512^{VI} compared with the number of segment 512^{IV}. The length of each segment 512^{VI} can be identical to the length of each segment 512^{IV}, such that the total length L^{V} of the strut 508^{V} is greater than the length L^{VI} of the strut 508^{VI}, L^{V}>L^{VI}. In the illustrated example, since strut 508^{V} includes twice the number of segments 512 than strut 508^{IV}, it length can be about twice the length of the 3strut 508^{IV}, L^{V}≠2L^{VI}.

The term “about”, as used herein, refers to being in the range of 10% from a value. For example, L^{V} being about twice L^{VI} means that L^{V} can be in the range of 90%-110% of 2L^{VI}. The range is defined to compensate for the fact that the length of the strut 508 is not only the sum of lengths of all segments 512, but of the lengths of the connection portions 510 as well.

In some examples, struts 508 are the first-type struts, and struts 508^{VI} are the second-type struts. While the segments 512^{V} and 512^{IV} are shown to have a linear configuration with constant lengths in the illustrated examples, in alternative examples, segments of both types of struts can have curved configurations, such as curved segments 212^{I} or 212^{II} of struts 208^{I} or 208^{II} shown in FIGS. 4A-B, and/or may have varying lengths, such as the progressively varying lengths of subsequent segments 212^{I} or 212^{II} of struts 208^{I} or 208^{II}. In such cases, in some examples, the shape (e.g., degree of curvature) and/or length of segments 512^{VI}a, 512^{VI}b and 512^{VI}c, can be identical to that of segments 512^{V}d, 512^{V}e and 512^{V}f, respectively. For example, in some examples, L^{VI}a=L^{V}d, L^{VI}b=L^{V}e, and L^{VI}c=L^{V}f.

FIG. 12C shows a flattened projection of another example of a strut 508^{VII} comprising two segments: a proximal segment 512^{III}a and a distal segment 512^{III} b which is a curved segment. In some examples, as shown, the proximal segment 512^{VII}a can be a linear segment. Alternatively, the proximal segment can be curved (example not shown). However, if both the proximal segment and the distal segment are curved, the degree of curvature of the distal segment is higher than that of the proximal segment.

The distal segment 512^{VII} b may be curved such that the connection portion 510^{VII} at its distal end (defining the clamp apex 516 when a clamping portion 514 is assembled) is offset from a linear axis 18 passing through the centers of the connection portion 510^{VII} at both ends of the proximal segment 512^{VII}a by an offset distance G. According to some examples, the offset distance G is greater than the width W of the connection portions 510^{VII}, such that G>W. According to some examples, the offset distance G is greater than three times the width W of the connection portions 510^{VII}, such that G>3W. According to some examples, the offset distance G is greater than five times the width W of the connection portions 510^{VII}, such that G>5W. The distance G can be measured between the axis 18 and the center of the distal-most connection portion 510^{VII}, such as the center of its aperture 511.

In some examples, struts 508^{VII} are the third-type struts. In some examples, the number of second-type struts 508^{VI} is identical to the number of third-type struts 508^{VII}. The outflow apices 524 can be formed by first-type struts 508^{V} interconnected with other first-type struts 508^{V} or with third-type struts 508^{VII} but not with second-type struts 508^{VI}. The inflow apices 526 can be formed by first-type struts 508^{V} interconnected with other first-type struts 508^{V} or with second-type struts 508^{VI}, but not with third-type struts 508^{VII} Each second-type strut 508^{VI} along the frame 506 is diagonally aligned with a corresponding proximal segment 512^{VII}a of a third-type strut 508^{VII}, such that an axis 18 extending through the centers of all connection portions 510^{VI} of the second-type struts 508^{VI}, can continuously pass through the centers of the connection portions 510^{VII} on both ends of the proximal segment 512^{III}a aligned therewith.

As shown in FIGS. 11A-B for an example of a prosthetic valve 500^{a}, pairs of third-type struts 508^{VII} are interconnected at corresponding clamp apices 516, such that each interconnected pair of distal segments 512^{VII}b defines a substantially V-shaped clamping portion, which is interconnected at non-apical junctions 522 (such as proximal-most non-apical junctions 522a) with first-type struts 508^{V}. The length of the second-type struts 508^{VI}, dictated by the number of segments 512^{VI}, is set to allow the second-type struts 508^{VI} to extend proximally from the inflow apices 526 and terminate at non-apical junctions 522 that are lateral to the third-type struts 508^{VII}, thereby preventing any interference or contact between the second-type struts 508^{VI} and the third-type struts 508^{VII}, and in particular, prevent contact or interference with the clamping portions 514 at any configuration, including the crimped or compressed configuration.

According to some examples, a mechanically expandable prosthetic valve 500 comprises a plurality of expansion and locking assemblies 560, configured to facilitate expansion or contraction of the valve 500, and in some instances, to lock the valve 500 at selected expanded state, preventing unintentional compression thereof. While FIGS. 11A-B illustrate three expansion and locking assemblies 560, mounted to the frame 506, and optionally equiangularly spaced from each other around an inner surface thereof, it should be clear that a different number of expansion and locking assemblies 560 may be utilized, that the expansion and locking assemblies 560 can be mounted to the frame around its outer surface, and that the circumferential spacing between expansion and locking assemblies 560 can be unequal.

The expansion and locking assemblies 560 can be implemented according to any of the examples described hereinabove for any expansion and locking assembly 260. For example, an expansion and locking assembly 560 can include a ratcheting-type mechanism similar to that described for expansion and locking assemblies 360, and may include an outer member 562 defining an outer member lumen 564, secured to a component of the valve 500, such as the frame 506, at a first location, and an inner member 576 secured to a component of the valve 500, such as the frame 506, at a second location, axially spaced from the first location.

The inner member 576 extends between an inner member proximal end portion 578 and an inner member distal end portion 580. The inner member 576 comprises an inner member coupling extension 584 extending from its distal end portion 580, which may be formed as a pin extending radially outward from the distal end portion 580, configured to be received within respective apertures 511 of struts 508 intersecting at a junction 520. The inner member 576 may further comprise a linear rack having a plurality of ratcheting teeth 586 along at least a portion of its length. According to some examples, inner member 576 further comprises a plurality of ratcheting teeth 586 along a portion of its outer surface.

The outer member 562 comprises an outer member proximal end portion 566 defining a proximal opening of its lumen 564, and an outer member distal end portion 368 defining a distal opening of its lumen 564. The outer member 562 can further comprise an outer member coupling extension 570 extending from its proximal end portion 566, which may be formed as a pin extending radially outward from the external surface of the proximal end portion 566, configured to be received within respective apertures 511 of struts 508 intersecting at a junction 520.

The outer member 562 can further comprise a spring-biased arm 572, attached to or extending from one sidewall of the outer member 562, and having a tooth or pawl 574 at its opposite end, biased inward toward the inner member 576 when disposed within the outer member lumen 564.

At least one of the inner or outer member 576 or 562, respectively, is axially movable relative to its counterpart. The expansion and locking assembly 560 in the illustrated example, comprises a ratchet mechanism or a ratchet assembly, wherein the pawl 574 is configured to engage with the teeth 586 of the inner member 576. The spring-biased arm 572 can comprise an elongate body terminating in a pawl 574 in the form of a locking tooth, configured to engage the ratcheting teeth 586 of the inner member 576. The pawl 574 can have a shape that is complimentary to the shape of the teeth 586, such that the pawl 574 allows sliding movement of the inner member 576 in a first direction relative to the spring-biased arm 572 (e.g., a proximally oriented direction) and resists sliding movement of the inner member 576 in the opposite second direction (e.g., a distally oriented direction) when the pawl 574 is in engagement with one of the teeth 586.

The arm 572 can be biased inwardly such that the pawl 574 is resiliently retained in a position engaging one of the teeth 586 of the inner member 576. In the illustrated example, the spring-biased arm 572 is configured as a leaf spring. In some examples, the spring-biased arm 572 can be integrally formed with the outer member 562, in other examples, the spring-biased arm 572 can be separately formed and subsequently coupled to the outer member 562. The biased configuration of the arm 572 ensures that under normal operation, the pawl 574 stays engaged with the teeth 586 of the inner member 576.

The spring-biased arm 572 can be formed of a flexible or resilient portion of the outer member 562 that extends over and contacts, via its pawl 574, an opposing side of the outer surface of the inner member 576. According to some examples, the spring-biased arm 572 can be in the form of a leaf spring that can be integrally formed with the outer member 562 or separately formed and subsequently connected to the outer member 562. The spring-biased arm 572 is configured to apply a biasing force against the outer surface of the inner member 576, so as to ensure that under normal operation, the pawl 574 stays engaged with the ratcheting teeth 586 of the inner member 576.

According to some examples, the first location can be positioned at or adjacent to the outflow end 502, and the second location can be positioned at or adjacent to the inflow end 504. In the illustrated example, the outer member 562 is secured to a proximal-most non-apical junction 522a which is distal to the outflow apices 524 or the outflow end 502, via outer member coupling extension 570, and the inner member 576 is secured to a distal-most non-apical junction 522b which is proximal to the inflow apices 526 or the inflow end 504, via inner member coupling extension 584.

It is to be understood that while the illustrated examples are for an expansion and locking assembly 560 secured to a proximal-most non-apical junction 522a serving as the first location, and to a distal-most non-apical junction 522b serving as the second location, in other implementations, the expansion and locking assembly 560 (or any other type of an expansion and locking assembly 260) can be secured to other junctions. For example, the expansion and locking assembly can be secured to an outflow apex 524 via the outer member coupling extension 570, serving as the first location, and to an opposing inflow apex 526 along the same column of cells, via the inner member coupling extension 584, serving as the second location.

The expansion and locking assemblies can be releasably coupled to actuators or actuation assemblies of a delivery apparatus (not shown). For example, an actuator in the form of an axially movable wire or cable can be releasably attached to the inner member proximal end portion 578, such as via threaded portion 582 or any other type of engagement, and may be controlled via mechanisms in the handle of the delivery apparatus to move proximally and pull the inner member 576 therewith, optionally while a sleeve disposed around the actuator is pressed against the outer member proximal end 568 to provide a counter-force.

In the example illustrated in FIG. 11B, the inner member coupling extension 584 extends through apertures 511 in two struts 508 interconnected at a distal non-apical junction 522b, while the outer member coupling extension 570 extends through aperture 511 in two struts 508 interconnected at a proximal non-apical junction 522a. As such, when the inner member 576 is moved axially, for example upward (or in a proximally oriented direction), within the outer member lumen 564, the inner member coupling extension 584 moves along with the inner member 576, thereby causing the portion to which the inner member coupling extension 584 is attached to move axially as well, which in turn causes the frame 506 to foreshorten axially and expand radially.

The struts 508 to which the inner member coupling extension 584 is connected are free to pivot relative to the coupling extension 584 and to one another as the frame 506 assumes various expansion diameters. In this manner, the inner member coupling extension 584 serves as a fastener that forms a pivotable connection between those struts 508. Similarly, struts 508 to which the outer member coupling extension 570 is connected are also free to pivot relative to the coupling extension 570 and to one another as the frame 506 assumes various expansion diameters. In this manner, the outer member coupling extension 570 also serves as a fastener that forms a pivotable connection between those struts 508.

As mentioned above, when the pawl 574 of the spring-biased arm 572 is engaged with the ratcheting teeth 586, the inner member 576 can move in one axial direction, such as the upward or proximally oriented direction, but cannot move in the opposite axial direction. This ensures that while the pawl 574 is engaged with the ratcheting teeth 586, the frame 506 can be further radially expanded but cannot be radially compressed. In this manner, the actuation mechanism also serves as a locking mechanism of the prosthetic valve 500.

Once the desired diameter of the prosthetic valve 500 is reached, the actuators of the delivery apparatus may be rotated and unscrewed from the inner members 576, enabling the delivery apparatus to be pulled away, and retracted, from the patient's body, leaving the prosthetic valve 100 implanted in the patient. The patient's native anatomy, such as the native aortic annulus in the case of transcatheter aortic valve implantation, may exert radial forces against the prosthetic valve 500 that would strive to compress it. However, the engagement between the pawl 574 of the spring-biased arm 572 and the ratcheting teeth 586 of the inner member 576 prevents such forces from compressing the frame 506, thereby ensuring that the frame 506 remains locked in the desired expanded diameter.

According to some examples, the length L^{VII}a of the proximal segment 512^{VII}a can be identical to that of any one of the segments 512^{V} of first-type strut 508^{V} and/or any one of the segments 512^{II} of second-type strut 508^{VI}, such that L^{VII}a=L^{V}(i)=L^{VI} (i), wherein in one example, L^{V}(i) can be any one of L^{V}a to L^{V}f, and L^{VI} (i) can be any one of L^{VI}a to L^{VI}c.

According to some examples, the projected length L^{VII}b of the distal segment 512^{VII}b is greater than the length L^{VII}a of proximal segment 512^{VII} a, such that L^{VII}b>L^{VII}a. The projected length L^{VII}b may be defined as the distance of a linear projection of the distal segment 512^{VII}b along the axis 18 passing through the connection portions 510^{VII} at both ends of the proximal segment 512^{III} a. Since second-type struts 508^{VI} terminate laterally to the clamping portion 514^{a}, the clamping portion 514^{a} is disposed along a region which is devoid of cells, allowing both segments 512^{II}b and the clamp apex 516a to extend beyond the axial or vertical height of a cell, without interference. In particular, the clamping portion 514^{a}, in some examples, does not cross any other struts of the frame 506^{a} in any configuration, including in the compressed configuration.

In some examples, the projected length of the distal segment 512^{VII}b is shorter than twice the length of proximal segment 512^{VII} a, such that L^{VII}b<2L^{VII} a. Such a configuration, as shown for frame example 506^{a} of valve 500^{a} in FIGS. 11A-B, can potentially ensure that the distal segments 512^{VII}b defining the clamping portion 514^{a} do not cross with or extend over any other strut 508 of the frame 506^{a} at any diameter the valve 500^{a} can assume, including the crimped configuration. Stated otherwise, the clamp apex 516^{a} is always proximal to the next distally aligned junction 520, including in the crimped configuration.

In some examples, the frame 506 can be configured such that it has a barrel-shaped profile when in the compressed configuration, and an hourglass-shaped profile when in the expanded configuration. As used herein, the term “barrel-shaped profile” means that a central portion of the frame 506 is offset radially outwardly from the inflow end 504 and the outflow end 502 with respect to the longitudinal axis 10 such that a diameter of the central portion of the frame 506 is greater than the diameters of the inflow and outflow ends. As used herein, the term “hourglass-shaped profile” means that the central portion of the frame 506 is offset radially inwardly from the inflow end 504 and from the outflow end 502 of the frame relative to the longitudinal axis 10 such that the diameter of the central portion is less than the diameters of the inflow and outflow ends.

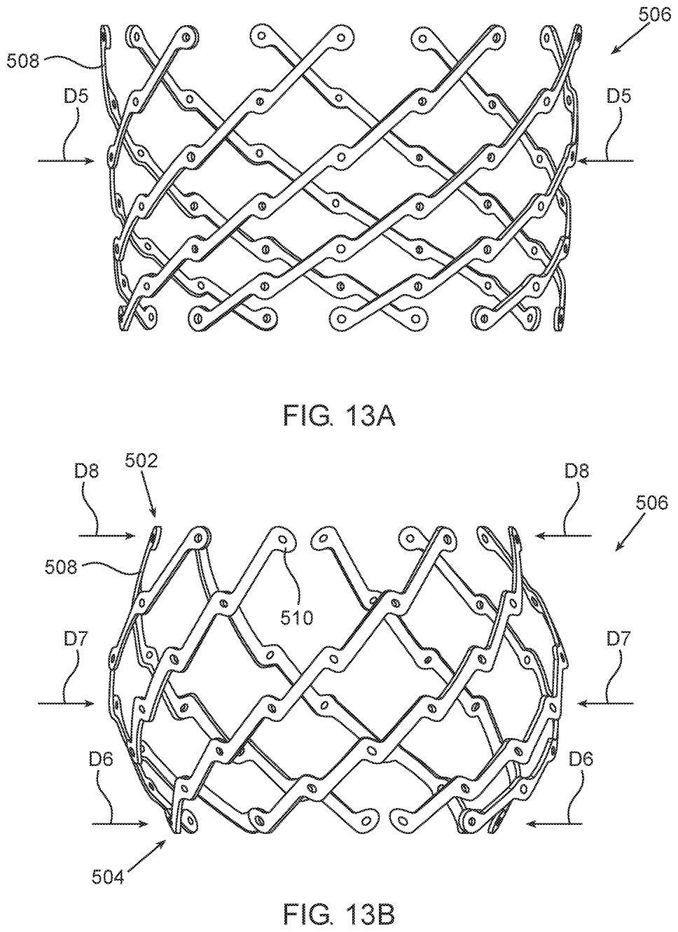

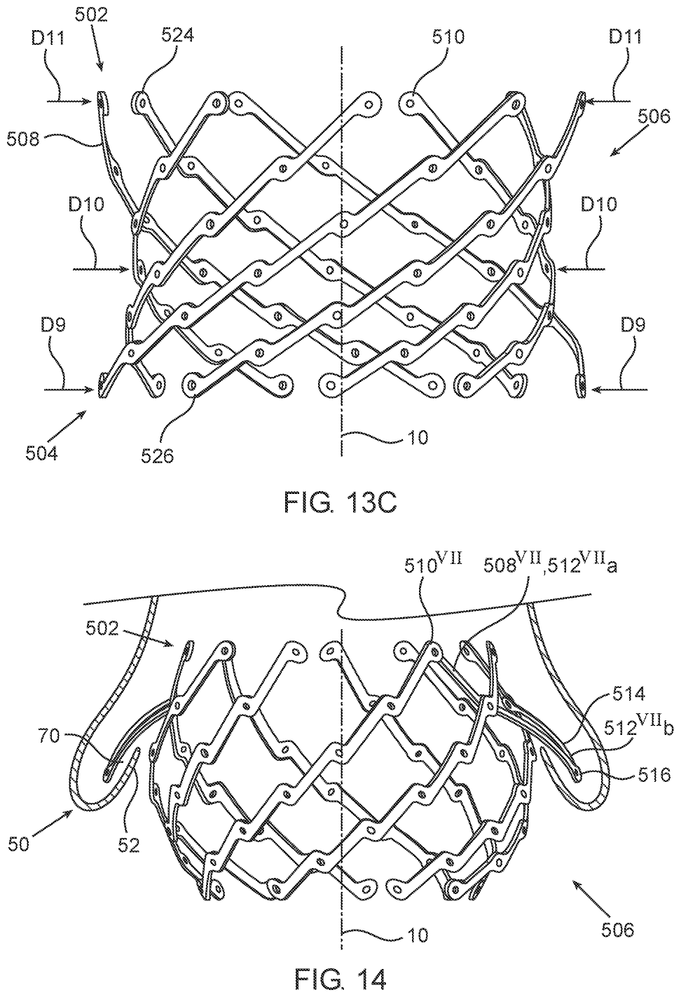

While the valve 500^{a}, as shown in FIGS. 11A-B, is composed of two layers of struts 508 interconnected with each other, FIG. 13A illustrates only one layer of struts 508 (e.g., an outer layer of the struts) of an example of a valve 500 that includes, for purposes of illustration, only a single type of struts 508, such as first-type struts 508^{V}. In some examples, the struts 508 can be cut (e.g., laser cut) from a tube such that the struts 508 are curved, and have a radius corresponding to a radius of the tube from which the struts were cut. Thus, the frame 506 has a “natural” diameter D5 corresponding to the diameter of the tube from which the struts 508 were cut. In other examples, the struts 508 can be cut from sheet stock and bent to the desired curvature.

In some examples, any of the first-type struts 508^{V}, second-type struts 508^{VI}, and third-type struts 508^{VII}, can be cut from one or more tubes such that the struts are curved to a natural diameter of the tube from which the struts were cut. In some examples, the natural diameter of the first-type struts 508^{V} and the second-type struts 508^{VI} is identical, while the natural diameter of the third-type struts 508^{VII} may be larger, the same, or smaller than the natural diameter D5 of the first-type struts 508^{V} and the second-type struts 508^{VI}, depending upon the particular configuration.

When the prosthetic valve 500 is crimped to the radially compressed configuration, which is less than the natural diameter D5 of the frame, the central portions of the struts 508, and in particular, of first-type struts 508^{V} and/or the second-type struts 508^{VI}, can tend to bow radially outwardly such that the frame 506 has a barrel-shaped profile, as shown in FIG. 13B. More specifically, as the diameter of the frame 506 is reduced, the connection portions 510 at the inflow end 504 and the outflow end 502 can be positioned radially inwardly of the central portions of the struts 508 such that the inflow apices 526 and the outflow apices 524 are located closer to the longitudinal axis 10 than the central portions of the struts. In this manner, a diameter D6 of the inflow end 504 and a diameter D8 of the outflow end 502 can be smaller than a diameter D7 of the central portion of the frame 506.

In other words, the connection portions 510 at the inflow end 504 and the outflow end 502 can be located radially inwardly of the central portions of the struts 508 such that a concave side of the struts 508 (e.g., any of the first-type struts 508^{V} and the second-type struts 508^{VI}) is oriented toward the axis 10 when the diameter of the frame 506 is reduced below its natural diameter D5. A convex side of the struts 508 can be oriented away from the axis 10 when the diameter of the frame 506 is reduced below its natural diameter D5.

Conversely, when the prosthetic valve 500 is expanded to the expanded configuration, the frame 506 can be expanded beyond its natural diameter D5 such that the frame has an hourglass-shaped profile, as shown in FIG. 13C. With reference to FIG. 13C, the connection portions 510 at the inflow end 504 and the outflow end 502 can be positioned radially outwardly of the central portions of the struts 508 such that the inflow apices 526 and the outflow apices 524 are located farther away from the longitudinal axis 10 than the central portions of the struts 508. In this manner, a diameter D9 of the inflow end 504 and a diameter D11 of the outflow end 502 can be larger than a diameter D10 of the central portion of the frame 506. In other words, although a concave side of the struts 508 is still oriented toward the axis 10, the connection portions 510 at the inflow end 504 and the outflow end 502 can be located radially outwardly of the central portions of the struts when the diameter of the frame 506 is expanded beyond its natural diameter D5.

The fully assembled frame 506 including both layers of struts 508 can exhibit the shapes described above when in the compressed and expanded configurations, although the degree of the barrel-shaped profile and the hourglass-shaped profile achieved can vary due to constraints imposed by the junctions 520 and the opposite helicity of struts in each layer. As shown in FIGS. 13B-C, a length dimension of the frame 506 (e.g., in the direction of axis 10) can also shorten as the frame is expanded.

The variation in the radial position of the inflow and outflow ends 504, 502 with respect to the central portion of the frame 506 can be utilized to move the clamping portions 514 between the open and closed positions. For example, FIG. 14 illustrates the frame 506 (shown with a single layer of struts, for clarity) in a partially radially compressed configuration in a native heart valve 50 in which the frame has a barrel-shaped profile. In the position shown in FIG. 14, because the proximal connection portions 510^{VII} of the third-type struts 508^{VII} of the clamping portions 514 are coupled to the outflow end 502, the clamp apices 516 can be located radially outwardly of the proximal connection portions 510^{VII}. In this manner, the clamping portions 514 can define respective leaflet-receiving regions 70 between the segments 512^{VII}b and the surface of the frame 506 defined by the first-type struts 508^{V} and the second-type struts 508^{VI} (second type struts are omitted from view in FIG. 14 for clarity). The leaflet-receiving regions 70 can be configured to receive leaflets 52 of the native heart valve 50 during implantation. In some examples, the segments 512^{VII} b can also be bowed or curved such that a concave side of the clamping portions 514 is oriented radially inwardly toward the longitudinal axis 10, and a convex side of the clamping portions 514 is oriented radially outward away from the longitudinal axis 10.

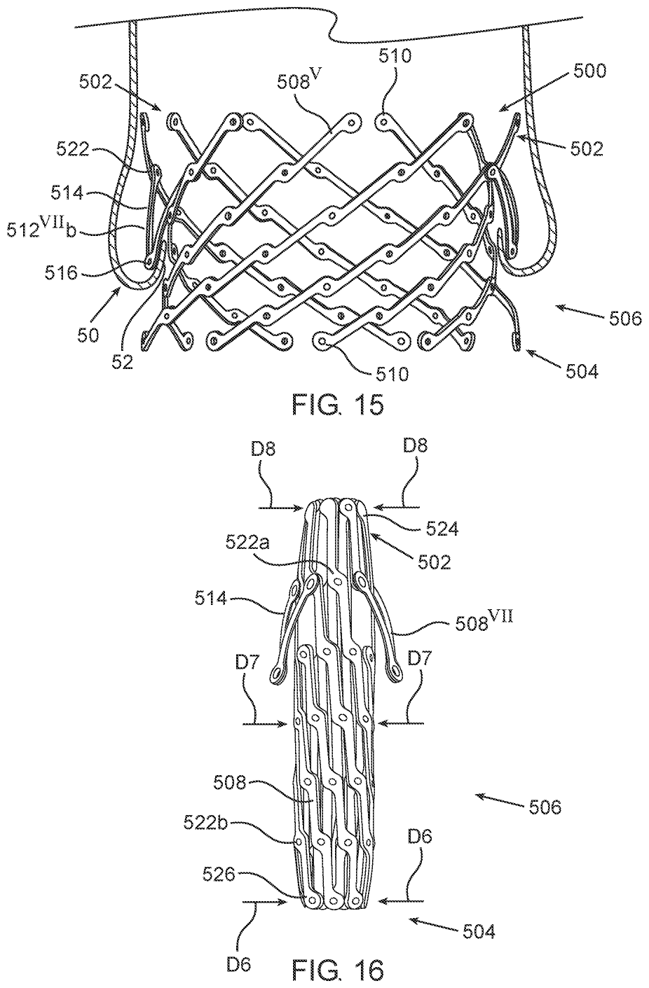

Conversely, when the prosthetic valve 500 is expanded to its functional size, the frame 506 can assume the hourglass-shaped profile illustrated in FIG. 15. As described above, as the frame 506 is expanded, the connection portions 510 at the inflow end 504 and the outflow end 502 can be positioned radially outwardly of the central portions of the struts 508. Stated otherwise, the inflow apices 526 and the outflow apices 524 are offset radially outwardly to non-apical junctions 522. This can cause the clamping portions 514 to move (e.g., by pivoting around proximal-most non-apical junctions 522a) to the closed position in which the clamp apices 516 can be adjacent or at the level of the surface of the remainder of frame 506. In this manner, the clamping portions 514 can clamp, grip, or clip the native leaflets 52 of heart valve 50 against the inner surfaces of the clamping portions 514.

In some examples. as shown in FIG. 15, in the radially expanded configuration of the frame 506 in which the clamping portions 514 are in the closed position, the segments 512^{VII}b can be slightly bowed to match the shape of the frame 506. In certain examples, the clamping portions 514 can include any of a variety of atraumatic coverings, such as woven or non-woven fabric, any of various electrospun coatings, and the like.

The clamping portions 514 described herein can provide significant advantages over known prosthetic valve docking mechanisms. For example, because the clamping portions are part of the struts 508 defining the frame 506, a separate docking member and the associated delivery apparatus are not required. Additionally, because the clamping portions can transition between the open and closed positions by motion of the frame 506 between the compressed and expanded configurations, the clamping portions can be easily reopened and the prosthetic valve 500 repositioned until the clinician is satisfied with the placement of the prosthetic valve.

An associated advantage of the mechanically expandable frame 506 is that a balloon is not required to expand the frame to its functional size and, thus, there is no occlusion of blood flow during valve expansion.

Additionally, although the illustrated configuration is adapted for implantation in the aortic valve, the frame and leaflet clamps can also be configured for implantation in the mitral valve and/or the tricuspid valve. For example, by reversing the clamping portions 514 such that the third-type struts 508^{CII} extend proximally from the inflow end 504, the leaflet clamping portions can be configured for use with the mitral valve and/or the tricuspid valve.

FIG. 16 illustrates the frame 506 crimped to the compressed configuration for delivery. In the compressed configuration, the first-type struts 508^{V} and/or the second-type struts 508^{VI} can curve radially inwardly beginning from about the location of the distal-most non-apical junctions 522b and moving in an upstream direction toward the inflow apices 526 at the inflow end 504 of the frame. Likewise, the first-type struts 508^{V} and/or the third-type struts 508^{VII} can curve radially inwardly beginning from about the location of the proximal-most non-apical junctions 522a and moving in a downstream direction toward the outflow apices 524 at the outflow end 502 of the frame.

Thus, the inflow end 504 at the level of the inflow apices 526 can have a diameter D6, and the outflow end 502 at the level of the outflow apices 524 can have a diameter D8 that may be approximately equal to the diameter D6. The diameters D6 and D8 can both be less than a diameter D7 of the central portion of the frame 506. For example, in some examples, the frame 506 can have the diameter D7 from approximately the level of the distal-most non-apical junctions 522b to approximately the level of the proximal-most non-apical junctions 522a such that the portion of the frame that includes the first-type struts and the second-type struts has a barrel-shaped profile. Because the first-type struts 508^{V} and/or the third-type struts 508^{VII} curve radially inward beginning at the proximal-most non-apical junctions 522a, the clamping portions 514 can be angled away from the rest of the frame in the open position.

A mechanically expandable prosthetic valve with leaflet clamps, exhibiting similar advantages, has been disclosed in US Patent Application Publication No. US 2019/0328518, which is incorporated herein by reference. The clamps of the previously disclosed valve are separate V-shaped components that are attached to the frame, such that once the valve is compressed to the crimped configuration, and optionally placed within a capsule or a catheter disposed there-over for delivery to the implantation site, the added clamps are overlayed over the external surface of the frame, resulting in an added layer of struts.

An important design parameter of a transcatheter prosthetic heart valve is the diameter of the folded or crimped profile. The diameter of the crimped profile is important because it directly influences the clinician's ability to advance the prosthetic valve through the femoral artery or vein. More particularly, a smaller profile allows for treatment of a wider population of patients, with enhanced safety. While the clamping portions 514 are biased away from the rest of the frame 506 in the crimped state shown in FIG. 16, once the valve is inserted into a capsule, or is covered by a distal portions of a delivery catheter, the clamping portions are forced radially inward by the encapsulating walls and may be disposed in-line with the remainder to the frame 506, for example such that the overall diameter at the level of clamp apices 516a does not exceed the diameter D7 of the central portion of the remainder of the frame 506.

The shorter second-type struts 508^{VI}, terminating proximal to the outflow end of the valve and sideways to the clamping portions 514, create a space into which the third-type struts 508^{VII} may be pressed (for example when placed within a capsule or a catheter in a crimped configuration) without intersecting with the second-type struts 508^{VI}. Thus, in contrast to the previously disclosed clamps, the clamping portion 514^{a} of prosthetic valve 500^{a} can reside at the level of the remainder of the frame 506 when the valve is delivered in a crimped configuration within a capsule or a catheter, to the implantation site. In other words, the distal segments 512^{VII}b of the third-type struts 508^{VII} are dimensioned such that the clamping portions 514^{a} can reside at the level of the surface of the remainder of the frame, defined by the first-type struts and the second-type struts, without the clamp apices 516^{a} projecting radially outward from laterally-adjacent non-apical junctions on both sides thereof.

This results in a lower overall crimped profile, which permits the clinician to more easily navigate the delivery apparatus (including the prosthetic valve 500) through a patient's vasculature to the treatment location. The lower profile of the crimped valve is particularly helpful when navigating through portions of the patient's vasculature which are particularly narrow, such as the iliac artery.

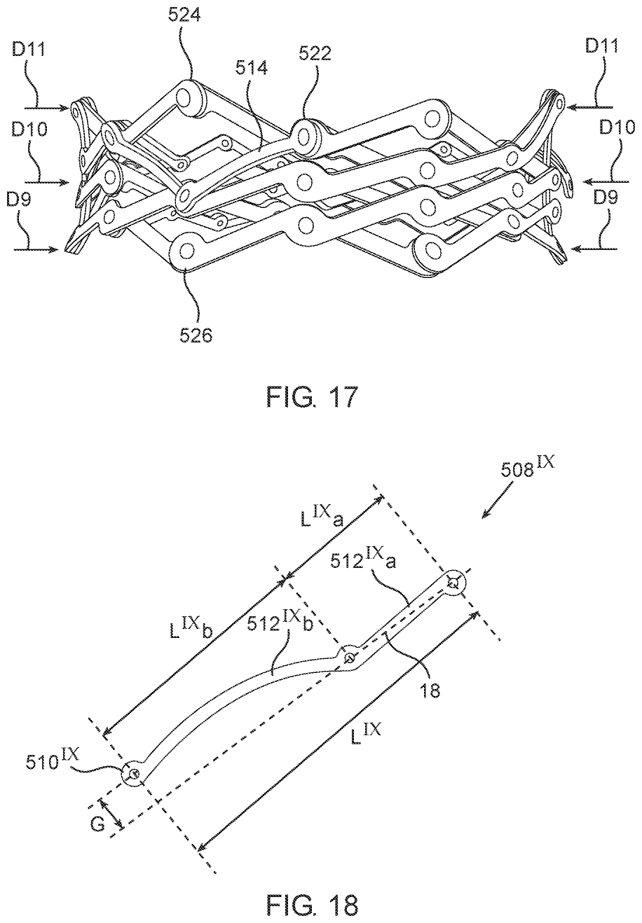

FIG. 17 shows the frame 506 expanded to the expanded configuration. The frame 506, and in particular such that the portion of the frame that includes the first-type struts and the second-type struts, can have an hourglass-shaped profile in which the inflow apices 526 and the outflow apices 524 are disposed radially outwardly of the non-apical junctions 522 therebetween. In this configuration, the frame can have a diameter D9 at the inflow apices 526 at the inflow end 504, and a diameter D11 at the outflow apices 524 at the outflow end 502. The central portion of the frame can have a minimum diameter D10. Because the struts 508 twist, curve, or flare radially outwardly at the inflow and outflow ends of the frame, the clamping portions 514 can be angled inwardly toward the frame. This can allow the clamping portions 514 to clamp the leaflets of a native heart valve, as described above.

FIG. 18 shows a flattened projection of another example of a strut 508^{IX} comprising two segments: a proximal segment 512^{IX}a and a distal segment 512^{IX}b. The strut 508^{IX} can be identical to any examples of the strut 508^{VII} except that the projected length L^{I}b of the distal curved segment 512^{IX}b is greater than the length L^{VII}a of proximal segment 512^{VII}a. The length L^{IX}a of the proximal segment 512^{IX}a, which can be a linear segment, can be similar to that of any example of proximal segment 512^{III} a, such as being identical to that of any one of the segments 512^{V} of first-type strut 508^{V} and/or any one of the segments 512^{VI} of second-type strut 508^{VI}, while the projected length L^{IX}b of the distal curved segment 512^{IX}b is greater than the length L^{IX}a of the proximal segment 512^{IX}a, such that L^{IX}b>L^{IX}a.

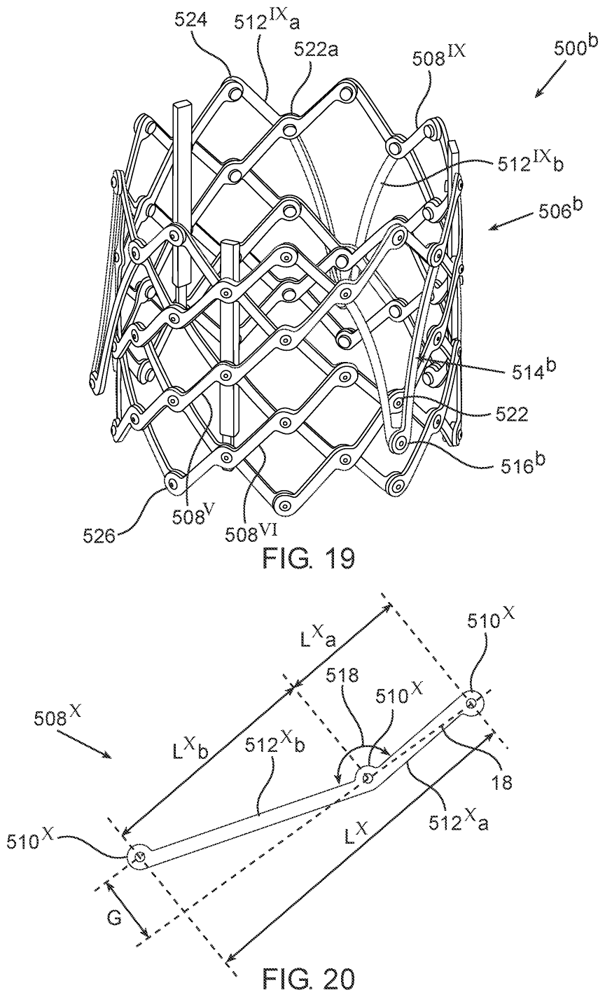

In some examples, struts 508^{Ix} are the third-type struts. FIG. 19 shows an example of a prosthetic valve 500^{b} which can be identical to prosthetic valve 500^{a}, including first-type struts 508^{V} and second-type struts 508^{VI} comprised within its frame 506^{b}, except that it includes third-type struts 508^{IX} and clamping portions 514^{b} comprised of distal segments 512^{IX}b thereof. In some examples, the projected length of the distal curved segment 512^{IX}b is greater than twice the length of proximal segment 512^{III} a, such that L^{IX}b>2L^{IX}a. In some examples, the projected length of the distal curved segment 512^{IX}b is greater than three times the length of proximal segment 512^{VII} a, such that L^{IX}b>3L^{IX}a. Such configurations, as shown for frame example 506^{b} of valve 500^{b}, can result in distal curved segments 512^{IX}b defining a clamping portion 514^{b} partially extending over other struts 508 of the frame 506^{b} in at least some of the diameters the valve 500^{b} can assume, such as the crimped configuration. Thus, in a closed position of the clamping portion 514^{b}, they may be partially overlayed over other struts 508 of the frame 506^{b}, particularly in a crimped configuration, when the clamping portions 514^{b} are pressed against the outer surface of first-type struts 508^{V} of the frame 506^{b} by a capsule or an encapsulating catheter.

While the crimped profile of the valve 500^{b} is larger than that of the valve 500^{a} at the level of the clamp apices 516a, some regions along the clamping portions 514^{b} still result in lower crimped profile when compared to previously disclosed valves with separate attachable leaflet clamps, due to the angled path of the segments 512^{IX}b from the clamp apices 516a radially inward toward their attachments at non-apical junctions to first-type struts 508^{V}. On the other hand, the longer clamping portions 514^{b} may result in a larger clamping area in which the native leaflets are clamped, potentially improving retention thereof. Thus, the longer clamping portions 514^{b} provide a compromise between the advantages conferred by the narrower crimping profile and the increased leaflets retention force.

FIG. 20 shows a flattened projection of another example of a strut 508^{X} comprising two segments: a proximal segment 512^{X}a and a distal segment 512^{X}b. The strut 508^{X} can be identical to any examples of strut 508^{VII} or strut 508^{IX}, except that the distal segment 512^{IX}b is a linear segment instead being a curved segment.

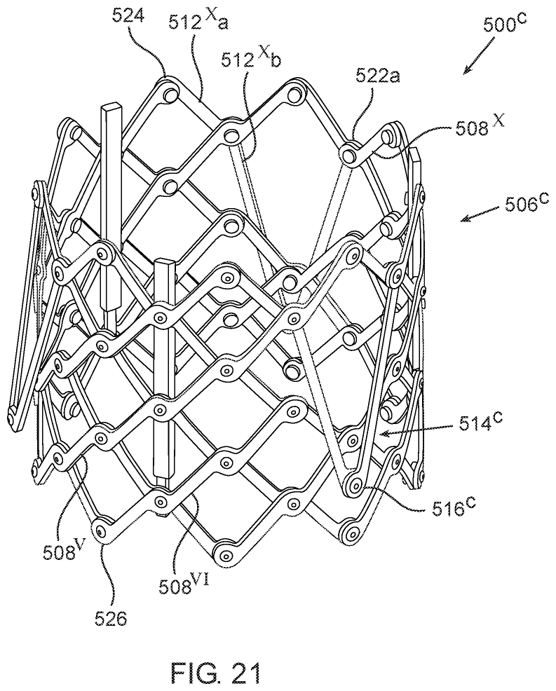

In some examples, struts 508^{X} are the third-type struts. FIG. 21 shows an example of a prosthetic valve 500^{c} which can be similar to prosthetic valve 500^{a} or prosthetic valve 500^{b}, including first-type struts 508^{V} and second-type struts 508^{VI} comprised within its frame 506^{b}, except that it includes third-type struts 508^{X} and clamping portions 514^{c} comprised of distal linear segments 512^{X}b thereof. The length of the linear distal segment 512^{X}b is preferably longer than that of the proximal segment 512^{X}a, such that the distal segment 512^{X}b is not linearly continuous with the proximal segment 512^{X}a, but is rather angled relative thereto, such as the configuration illustrated in FIGS. 20-21 showing an obtuse angle 518 formed between the two segments. According to some examples, the angle 518 formed between the proximal segment 512^{X}a and the distal segment 512^{X}b is not greater than 170 degrees.

While the example illustrated in FIG. 21 is for relatively long distal curved segments 512^{X}b defining a clamping portion 514^{c} partially extending over other struts 508 of the frame 506^{b}, such that the length L^{X}b can be similar to examples of the length L^{I}b, it is to be understood that other lengths are contemplated, such as a length L^{X}b of a distal segment 512^{X}b similar to examples of length L^{VII}b, which may result in clamping portions that are not overlayed over or crossing any other struts 508 of the frame.

It is to be understood that while prosthetic valves 500 are described herein for clamping native leaflets of a native heart valve, the valves 500 can be similarly utilized for clamping leaflets of leaflets of a host prosthetic valve instead of a native heart valve. For example, certain procedures may involve percutaneous implantation of a prosthetic heart valve within a malfunctioning, previously implanted heart-valve. In such cases, the guest valve 500 can include the clamping portions 514 utilized to clamp over the prosthetic leaflets of the previously implanted prosthetic valve, in the same manner described above in conjunction with FIGS. 14-15, mutatis mutandis.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve, comprising:
a frame movable between a range of diameters, the frame extending between an inflow end and an outflow end and comprising:
   a plurality of struts wherein each strut comprises a plurality of connection portions and segments extending between the connection portions, wherein the segments of each struts comprise a proximal-most segment and at least one subsequent segment, wherein the plurality of strut are pivotably interconnected at their connection portions, thereby defining a plurality of junctions, the plurality of junctions comprising a plurality of outflow apices along the outflow end, a plurality of inflow apices along the inflow end, and a plurality of non-apical junctions between the outflow end and the inflow end, and wherein the plurality of struts comprises:
      a plurality of first-type struts and a plurality of second-type struts;
wherein each strut is curved helically with respect to a longitudinal axis of the frame;
wherein the first-type strut is longer than the second-type strut; and
wherein the first-type struts extend distally from the outflow end, and wherein the second-type struts extend distally from non-apical junctions which are distal to the outflow end.

Example 2. The prosthetic valve of any example herein, particularly example 1, wherein each first-type strut comprises three segments, and wherein the proximal-most segment of the first-type strut is longer than any of its subsequent segments.

Example 3. The prosthetic valve of any example herein, particularly example 2, wherein the segments subsequent to the proximal-most segment of the first-type strut are having progressively shorter lengths.

Example 4. The prosthetic valve of any example herein, particularly any one of examples 2 or 3, wherein each second-type strut comprises three segments, wherein the proximal-most segment of the second-type strut is longer than any of its subsequent segments, and wherein the proximal-most segment of the first-type strut is longer than any of the segments of the second-type strut.

Example 5. The prosthetic valve of any example herein, particularly example 4, wherein the segments subsequent to the proximal-most segment of the second-type strut are having progressively shorter lengths.

Example 6. The prosthetic valve of any example herein, particularly any one of examples 2 or 3, wherein each second-type strut comprises two segments, wherein the proximal-most segment is longer than the subsequent segment.

Example 7. The prosthetic valve of any example herein, particularly any one of examples 1 to 6, wherein the struts are concave with respect to the outflow end.

Example 8. The prosthetic valve of any example herein, particularly any one of examples 1 to 7, wherein the frame has a draft angle that increases when the valve is expanded to a larger diameter of the outflow end.

Example 9. The prosthetic valve of any example herein, particularly any one of examples 1 to 8, wherein the first-type struts extend from the outflow apices to non-apical junctions.

Example 10. The prosthetic valve of any example herein, particularly example 9, wherein

Example 11. The prosthetic valve of any example herein, particularly any one of examples 1 to 10, wherein pairs of second-type struts intersect at the inflow apices and extend therefrom to non-apical junctions.

Example 12. The prosthetic valve of any example herein, particularly example 11, wherein the second-type struts extend from the inflow apices to proximal-most non-apical junctions.

Example 13. The prosthetic valve of any example herein, particularly any one of examples 1 to 12, further comprising a leaflet assembly mounted within the frame, the leaflet assembly comprising a plurality of leaflets configured to regulate flow through the prosthetic valve, wherein each leaflet comprises a pair of oppositely-directed tabs, and wherein the tabs of adjacent leaflets are paired to form commissures.

Example 14. The prosthetic valve of any example herein, particularly example 13, further comprising a plurality of support members, wherein each support member is attached to four struts defining a commissure cell along a first row of cells of the frame, and wherein each commissure is attached to a respective support member.

Example 15. The prosthetic valve of any example herein, particularly example 14, wherein each support member covers the entire area of the corresponding commissure cell.

Example 16. The prosthetic valve of any example herein, particularly any one of examples 1 to 15, wherein each segment of the first-type strut is curved with respect to a first lateral axis.

Example 17. The prosthetic valve of any example herein, particularly example 13, wherein the proximal-most segment of each first-type strut comprises a proximal portion extending from the outflow end, and wherein each commissure is attached to a pair of proximal portions.

Example 18. The prosthetic valve of any example herein, particularly example 17, wherein each pair of adjacent proximal portions defines a commissure angle that is not greater than 30 degrees at the maximal diameter of the prosthetic valve.

Example 19. The prosthetic valve of any example herein, particularly example 18, wherein the commissure angle is not greater than 15 degrees at the maximal diameter of the prosthetic valve.

Example 20. The prosthetic valve of any example herein, particularly example 18, wherein the commissure angle is not greater than 10 degrees at the maximal diameter of the prosthetic valve.

Example 21. The prosthetic valve of any example herein, particularly example 18, wherein the commissure angle is not greater than 5 degrees at the maximal diameter of the prosthetic valve.

Example 22. The prosthetic valve of any example herein, particularly any one of examples 17 to 21, wherein the length of the proximal portion is at least as great as a quarter of the length of the proximal-most segment of the first-type strut.

Example 23. The prosthetic valve of any example herein, particularly any one of examples 17 to 21, wherein the length of the proximal portion is at least as great as a third of the length of the proximal-most segment of the first-type strut.

Example 24. The prosthetic valve of any example herein, particularly any one of examples 17 to 21, wherein the length of the proximal portion is at least as great as half of the length of the proximal-most segment of the first-type strut.

Example 25. The prosthetic valve of any example herein, particularly any one of examples 1 to 24, wherein pairs of the first-type struts intersect at the outflow apices.

Example 26. The prosthetic valve of any example herein, particularly any one of examples 17 to 24, wherein each pair of adjacent proximal portions are distanced from each other at the outflow end.

Example 27. The prosthetic valve of any example herein, particularly example 26, wherein the distance between each pair of adjacent proximal portions is not greater than four times the thickness of the tab of the leaflet.

Example 28. The prosthetic valve of any example herein, particularly example 26, wherein the distance between each pair of adjacent proximal portions is not greater than twice the thickness of the tab of the leaflet.

Example 29. The prosthetic valve of any example herein, particularly any one of examples 1 to 28, wherein the plurality of non-apical junctions comprises a plurality of proximal-most non-apical junctions, wherein exactly three segments extend from each proximal-most non-apical junction.

Example 30. The prosthetic valve of any example herein, particularly any one of examples 1 to 29, wherein the plurality of non-apical junctions comprises a plurality of distal-most non-apical junctions, wherein exactly three segments extend from each distal-most non-apical junction.

Example 31. The prosthetic valve of any example herein, particularly any one of examples 1 to 30, wherein comprising a sewing ring that circumscribes the frame.

Example 32. The prosthetic valve of any example herein, particularly example 31, wherein the sewing ring comprises a ring insert and a cloth cover around the ring insert.

Example 33. The prosthetic valve of any example herein, particularly any one of examples 31 or 32, further comprising a skirt mounted on the outer surface of the frame, and wherein the sewing ring is sutured to the skirt.

Example 34. The prosthetic valve of any example herein, particularly any one of examples 1 to 33, further comprising a plurality of expansion and locking assemblies, wherein each expansion and locking assembly is secured to the frame at a first location and a second location that is axially spaced from the first location, and configured to facilitate expansion or contraction of the prosthetic valve by approximating the first and second locations to each other, or distancing them away from each other, respectively.

Example 35. The prosthetic valve of any example herein, particularly example 34, wherein the plurality of expansion and locking assemblies comprises three expansion and locking assemblies.

Example 36. The prosthetic valve of any example herein, particularly any one of examples 34 or 35, wherein each expansion and locking assembly comprises:
an outer member, secured to the frame at the first location, and comprising a spring-biased arm with a pawl; and
an inner member, secured to the frame at the second location spaced apart from the first location, the inner member extending at least partially into the outer member, the inner member comprising a plurality of ratcheting teeth;
wherein the spring-biased arm is biased toward the inner member; and
wherein engagement of the pawl with the ratcheting teeth allows movement in a first direction to allow axial foreshortening and radial expansion of the frame and prevents movement in a second direction to prevent radial compression of the frame.

Example 37. The prosthetic valve of any example herein, particularly any one of examples 34 or 35, wherein each expansion and locking assembly comprises:
a first anchor, secured to the frame at the first location, and defining a first anchor channel;
a second anchor, secured to the frame at the second location, and defining a second anchor threaded channel; and
a rod extending through the first anchor channel and the second anchor threaded channel;
wherein the rod is threaded through the second anchor threaded channel such that rotation of the rod causes corresponding axial movement of the second anchor toward or away from the first anchor, depending on the direction of rotation.

Example 38. A prosthetic valve, comprising:
a frame movable between a range of diameters, extending between an inflow end and an outflow end and comprising a first row of cells comprising a plurality of commissure cells, and a second row of cells;
wherein the number of cells in the second row of cells is higher than the number of cells in the first row of cells.

Example 39. The prosthetic valve of any example herein, particularly example 38, wherein the first row of cells comprises three cells, and wherein the second row of cells comprises six cells.

Example 40. The prosthetic valve of any example herein, particularly any one of examples 38 or 39, wherein the height of the first row of cells is greater than the height of the second row of cells.

Example 41. The prosthetic valve of any example herein, particularly any one of examples 38 to 40, further comprising a third row of cells, wherein the number of cells in the second row of cells is higher than the number of cells in the third row of cells.

Example 42. The prosthetic valve of any example herein, particularly example 41, wherein the third row of cells comprises three cells.

Example 43. The prosthetic valve of any example herein, particularly any one of examples 41 or 42, wherein the height of the second row of cells is greater than the height of the third row of cells.

Example 44. The prosthetic valve of any example herein, particularly any one of examples 38 to 43, wherein the frame has a draft angle that increases when the valve is expanded to a larger diameter of the outflow end.

Example 45. The prosthetic valve of any example herein, particularly any one of examples 38 to 44, further comprising a leaflet assembly mounted within the frame, the leaflet assembly comprising a plurality of leaflets configured to regulate flow through the prosthetic valve, wherein each leaflet comprises a pair of oppositely-directed tabs, and wherein the tabs of adjacent leaflets are paired to form commissures.

Example 46. The prosthetic valve of any example herein, particularly example 45, wherein further comprising a plurality of support members, wherein each support member is attached to a cell of the first row of cells, and wherein each commissure is attached to a respective support member.

Example 47. The prosthetic valve of any example herein, particularly example 46, wherein each support member covers the entire area of the corresponding cell it is attached to.

Example 48. The prosthetic valve of any example herein, particularly example 45, wherein each cell of the first row of cells comprises a pair of proximal portions extending from the outflow end, and wherein each commissure is attached to the corresponding pair of proximal portions.

Example 49. The prosthetic valve of any example herein, particularly example 48, wherein each pair of adjacent proximal portions defines a commissure angle that is not greater than 30 degrees at the maximal diameter of the prosthetic valve.

Example 50. The prosthetic valve of any example herein, particularly example 49, wherein the commissure angle is not greater than 15 degrees at the maximal diameter of the prosthetic valve.

Example 51. The prosthetic valve of any example herein, particularly example 49, wherein the commissure angle is not greater than 10 degrees at the maximal diameter of the prosthetic valve.

Example 52. The prosthetic valve of any example herein, particularly example 49, wherein the commissure angle is not greater than 5 degrees at the maximal diameter of the prosthetic valve.

Example 53. The prosthetic valve of any example herein, particularly any one of examples 38 to 52, wherein further comprising a sewing ring that circumscribes the frame.

Example 54. The prosthetic valve of any example herein, particularly example 53, wherein the sewing ring comprises a ring insert and a cloth cover around the ring insert.

Example 55. The prosthetic valve of any example herein, particularly any one of examples 53 or 54, further comprising a skirt mounted on the outer surface of the frame, and wherein the sewing ring is sutured to the skirt.

Example 56. The prosthetic valve of any example herein, particularly any one of examples 38 to 55, wherein further comprising a plurality of expansion and locking assemblies, wherein each assembly is secured to the frame at a first location and a second location that is axially spaced from the first location, and configured to facilitate expansion or contraction of the prosthetic valve by approximating the first and second locations to each other, or distancing them away from each other, respectively.

Example 57. The prosthetic valve of any example herein, particularly example 56, wherein the plurality of expansion and locking assemblies comprises three expansion and locking assemblies.

Example 58. The prosthetic valve of any example herein, particularly any one of examples 56 or 57, wherein each expansion and locking assembly comprises:
an outer member, secured to the frame at the first location, and comprising a spring-biased arm with a pawl; and
an inner member, secured to the frame at the second location spaced apart from the first location, the inner member extending at least partially into the outer member, the inner member comprising a plurality of ratcheting teeth;
wherein the spring-biased arm is biased toward the inner member; and
wherein engagement of the pawl with the ratcheting teeth allows movement in a first direction to allow axial foreshortening and radial expansion of the frame and prevents movement in a second direction to prevent radial compression of the frame.

Example 59. The prosthetic valve of any example herein, particularly any one of examples 56 or 57, wherein each expansion and locking assembly comprises:
a first anchor, secured to the frame at the first location, and defining a first anchor channel;
a second anchor, secured to the frame at the second location, and defining a second anchor threaded channel; and
a rod extending through the first anchor channel and the second anchor threaded channel;
wherein the rod is threaded through the second anchor threaded channel such that rotation of the rod causes corresponding axial movement of the second anchor toward or away from the first anchor, depending on the direction of rotation.

Example 60. A prosthetic valve, comprising:
a frame movable between a compressed configuration and an expanded configuration, the frame extending between an inflow end and an outflow end and comprising:
   a plurality of struts wherein each strut comprises a plurality of connection portions and segments extending between the connection portions, wherein the plurality of struts are pivotably interconnected at their connection portions, thereby defining a plurality of junctions, the plurality of junctions comprising a plurality of outflow apices along the outflow end, a plurality of inflow apices along the inflow end, and a plurality of non-apical junctions between the outflow end and the inflow end, and wherein the plurality of struts comprises:
      a plurality of first-type struts, a plurality of second-type struts, and a plurality of third-type struts;
wherein the first-type struts extend from the inflow end to the outflow end;
wherein the second-type struts extend from the inflow end to non-apical junctions distal to the outflow end;
wherein each third-type strut comprises a proximal segment and a distal segment, wherein pairs of distal segments of the third-type struts are interconnected at a clamp apices, each pair of interconnected distal segments defining a clamping portion;
wherein the clamping portions are movable between an open position corresponding to the compressed configuration of the frame and a closed position corresponding to the expanded configuration of the frame; and
wherein motion of the frame between the compressed configuration and the expanded configuration causes corresponding motion of the clamping portions between the open position and the closed position.

Example 61. The prosthetic valve of any example herein, particularly example 60, wherein each proximal segment of a third-type strut is connected to two first-type struts at an outflow apex and a non-apical junction.

Example 62. The prosthetic valve of any example herein, particularly any one of examples 60 or 61, wherein the clamp apices are free ends that are not connected to any of the first-type struts or the second-type struts, such that when the clamping portions are in the open position, the clamp apices are spaced radially outwardly from the remainder of the frame.

Example 63. The prosthetic valve of any example herein, particularly any one of examples 60 to 62, wherein the clamp apices are proximal to the inflow end in the compressed configuration.

Example 64. The prosthetic valve of any example herein, particularly any one of examples 60 to 63, wherein the number of second-type struts is identical to the number of third-type struts, and wherein each second-type strut is diagonally aligned with a corresponding proximal segment of a third-type strut.

Example 65. The prosthetic valve of any example herein, particularly any one of examples 60 to 64, wherein the segments of the first-type strut are linear.

Example 66. The prosthetic valve of any example herein, particularly example 65, wherein all of the segments of the first-type strut have identical lengths.

Example 67. The prosthetic valve of any example herein, particularly any one of examples 60 to 66, wherein the segments of the second-type strut are linear.

Example 68. The prosthetic valve of any example herein, particularly example 67, wherein all of the segments of the first-type strut have identical lengths.

Example 69. The prosthetic valve of any example herein, particularly any one of examples 60 to 68, wherein the number of segments of the first type strut is larger than the number of segments of the second type strut.

Example 70. The prosthetic valve of any example herein, particularly example 69, wherein the first-type strut comprises six segments, and wherein the second-type strut comprises three segments.

Example 71. The prosthetic valve of any example herein, particularly any one of examples 60 to 70, wherein all of the segments of the first-type strut and all of the segments of the second-type struts have identical lengths.

Example 72. The prosthetic valve of any example herein, particularly example 71, wherein the length of the proximal segment of the third-type strut is equal to the length of a proximal-most segment of the first-type strut.

Example 73. The prosthetic valve of any example herein, particularly any one of examples 60 to 72, wherein the proximal segment of the third-type strut is linear.

Example 74. The prosthetic valve of any example herein, particularly any one of examples 60 to 73, wherein none of the second-type struts contacts any of the third-type struts in any configuration of the frame.

Example 75. The prosthetic valve of any example herein, particularly any one of examples 60 to 74, wherein the inflow apices and the outflow apices are offset radially outward to the non-apical junctions in the expanded configuration, such that the portion of the frame that includes the first-type struts and the second-type struts has an hourglass-shaped profile.

Example 76. The prosthetic valve of any example herein, particularly example 75, wherein the inflow apices and the outflow apices are offset radially inward to the non-apical junctions in the compressed configuration, such that the portion of the frame that includes the first-type struts and the second-type struts has a barrel-shaped profile.

Example 77. The prosthetic valve of any example herein, particularly any one of examples 60 to 76, wherein the distal segment of the third-type strut has a projected length that is greater than the length of the proximal segment of the third-type strut.

Example 78. The prosthetic valve of any example herein, particularly example 77, wherein the projected length of the distal segment is shorter than twice the length of the proximal segment.

Example 79. The prosthetic valve of any example herein, particularly any one of examples 60 to 78, wherein the third-type struts are dimensioned such that the clamping portions can reside at the level of the surface defined by the first-type struts and the second-type struts.

Example 80. The prosthetic valve of any example herein, particularly example 77, wherein the projected length of the distal segment is greater than twice the length of the proximal segment.

Example 81. The prosthetic valve of any example herein, particularly example 80, wherein the projected length of the distal segment is greater than at least three times the length of the proximal segment.

Example 82. The prosthetic valve of any example herein, particularly any one of examples 60 to 81, wherein the distal segment of the third-type strut is curved.

Example 83. The prosthetic valve of any example herein, particularly any one of examples 60 to 81, wherein the distal segment of the third-type strut is linear.

Example 84. The prosthetic valve of any example herein, particularly example 83, wherein the proximal segment and the distal segment form an angle there-between that is not greater than 170 degrees

[symbol]

Example 85. The prosthetic valve of any example herein, particularly any one of examples 60 to 84, further comprising a leaflet assembly mounted within the frame, the leaflet assembly comprising a plurality of leaflets configured to regulate flow through the prosthetic valve, wherein each leaflet comprises a pair of oppositely-directed tabs, and wherein the tabs of adjacent leaflets are paired to form commissures.

Example 86. The prosthetic valve of any example herein, particularly any one of examples 60 to 85, further comprising a plurality of expansion and locking assemblies, wherein each expansion and locking assembly is secured to the frame at a first location and a second location that is axially spaced from the first location, and configured to facilitate expansion or contraction of the prosthetic valve by approximating the first and second locations to each other, or distancing them away from each other, respectively.

Example 87. The prosthetic valve of any example herein, particularly example 86, wherein the plurality of expansion and locking assemblies comprises three expansion and locking assemblies.

Example 88. The prosthetic valve of any example herein, particularly any one of examples 86 or 87, wherein each expansion and locking assembly comprises:
an outer member, secured to the frame at the first location, and comprising a spring-biased arm with a pawl; and
an inner member, secured to the frame at the second location spaced apart from the first location, the inner member extending at least partially into the outer member, the inner member comprising a plurality of ratcheting teeth;
wherein the spring-biased arm is biased toward the inner member; and
wherein engagement of the pawl with the ratcheting teeth allows movement in a first direction to allow axial foreshortening and radial expansion of the frame and prevents movement in a second direction to prevent radial compression of the frame.

It is appreciated that certain features of the invention, which are, for clarity, described in the context of separate examples, may also be provided in combination in a single example. Conversely, various features of the invention, which are, for brevity, described in the context of a single example, may also be provided separately or in any suitable sub-combination or as suitable in any other described example of the invention. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve, comprising:
a frame movable between a range of diameters, the frame extending between an inflow end and an outflow end and comprising:
a plurality of struts wherein each strut comprises a plurality of connection portions and segments extending between the connection portions, wherein the segments of each struts comprise a proximal-most segment and at least one subsequent segment, wherein the plurality of strut are pivotably interconnected at their connection portions, thereby defining a plurality of junctions, the plurality of junctions comprising a plurality of outflow apices along the outflow end, a plurality of inflow apices along the inflow end, and a plurality of non-apical junctions between the outflow end and the inflow end, and wherein the plurality of struts comprises:
a plurality of first-type struts and a plurality of second-type struts;
wherein each strut is curved helically with respect to a longitudinal axis of the frame;
wherein the first-type strut is longer than the second-type strut; and
wherein the first-type struts extend distally from the outflow end, and wherein the second-type struts extend distally from non-apical junctions which are distal to the outflow end.

2. The prosthetic valve of claim 1, wherein each first-type strut comprises three segments, and wherein the proximal-most segment of the first-type strut is longer than any of its subsequent segments.

3. The prosthetic valve of claim 2, wherein the segments subsequent to the proximal-most segment of the first-type strut are having progressively shorter lengths.

4. The prosthetic valve of claim 2 or 3, wherein each second-type strut comprises three segments, wherein the proximal-most segment of the second-type strut is longer than any of its subsequent segments, and wherein the proximal-most segment of the first-type strut is longer than any of the segments of the second-type strut.

5. The prosthetic valve of claim 4, wherein the segments subsequent to the proximal-most segment of the second-type strut are having progressively shorter lengths.

6. The prosthetic valve of claim 2 or 3, wherein each second-type strut comprises two segments, wherein the proximal-most segment is longer than the subsequent segment.

7. The prosthetic valve of any one of claims 1 to 6, wherein the struts are concave with respect to the outflow end.

8. The prosthetic valve of any one of claims 1 to 7, wherein the first-type struts extend from the outflow apices to non-apical junctions.

9. The prosthetic valve of any one of claims 1 to 8, wherein pairs of second-type struts intersect at the inflow apices and extend therefrom to non-apical junctions.

10. The prosthetic valve of any one of claims 1 to 9, further comprising a leaflet assembly mounted within the frame, the leaflet assembly comprising a plurality of leaflets configured to regulate flow through the prosthetic valve, wherein each leaflet comprises a pair of oppositely-directed tabs, and wherein the tabs of adjacent leaflets are paired to form commissures.

11. The prosthetic valve of claim 10, further comprising a plurality of support members, wherein each support member is attached to four struts defining a commissure cell along a first row of cells of the frame, and wherein each commissure is attached to a respective support member.

12. The prosthetic valve of claim 10, wherein the proximal-most segment of each first-type strut comprises a proximal portion extending from the outflow end, and wherein each commissure is attached to a pair of proximal portions.

13. The prosthetic valve of claim 12, wherein each pair of adjacent proximal portions defines a commissure angle that is not greater than 30 degrees at the maximal diameter of the prosthetic valve.

14. The prosthetic valve of any one of claims 1 to 13, wherein pairs of the first-type struts intersect at the outflow apices.

15. The prosthetic valve of any one of claims 1 to 14, further comprising a sewing ring that circumscribes the frame.

16. The prosthetic valve of claim 15, further comprising a skirt mounted on the outer surface of the frame, and wherein the sewing ring is sutured to the skirt.

17. The prosthetic valve of any one of claims 1 to 16, further comprising a plurality of expansion and locking assemblies, wherein each expansion and locking assembly is secured to the frame at a first location and a second location that is axially spaced from the first location, and configured to facilitate expansion or contraction of the prosthetic valve by approximating the first and second locations to each other, or distancing them away from each other, respectively.

18. The prosthetic valve of claim 17, wherein the plurality of expansion and locking assemblies comprises three expansion and locking assemblies.

19. The prosthetic valve of claim 17 or 18, wherein each expansion and locking assembly comprises:
an outer member, secured to the frame at the first location, and comprising a spring-biased arm with a pawl; and
an inner member, secured to the frame at the second location spaced apart from the first location, the inner member extending at least partially into the outer member, the inner member comprising a plurality of ratcheting teeth;
wherein the spring-biased arm is biased toward the inner member; and
wherein engagement of the pawl with the ratcheting teeth allows movement in a first direction to allow axial foreshortening and radial expansion of the frame and prevents movement in a second direction to prevent radial compression of the frame.

20. The prosthetic valve of claim 17 or 18, wherein each expansion and locking assembly comprises:
a first anchor, secured to the frame at the first location, and defining a first anchor channel;
a second anchor, secured to the frame at the second location, and defining a second anchor threaded channel; and
a rod extending through the first anchor channel and the second anchor threaded channel;
wherein the rod is threaded through the second anchor threaded channel such that rotation of the rod causes corresponding axial movement of the second anchor toward or away from the first anchor, depending on the direction of rotation.
